(19) United States
(12) Patent Application Publication
(10) Pub. No.: US 2025/0032488 A1
(43) Pub. Date: Jan. 30, 2025

(54) METHODS OF TREATMENT USING LOU064

(71) Applicant: NOVARTIS AG, Basel (CH)

(72) Inventors: Souvik BHATTACHARYA, Weymouth, MA (US); Bruno BIETH, Saint-Louis (FR); Bruno CENNI, Sissach (CH); Gordon GRAHAM, Huningue (FR); Michael JUHNKE, Basel (CH); Monica LIGUEROS-SAYLAN, Basking Ridge, NJ (US); Dan Dragos MIHAILESCU, Rixheim (FR); Karin RAPP, Lörrach (DE); Kim-Hien Sin, Saint-Louis (FR); Bridget Danielle STUART, Rockwall, TX (US)

(21) Appl. No.: 18/715,832

(22) PCT Filed: Dec. 12, 2022
(86) PCT No.: PCT/IB2022/062046

§ 371 Date: Jun. 3, 2024

Related U.S. Application Data
(60) Provisional application No. 63/289,273, filed on Dec. 14, 2021.
(60) Provisional application No. 63/290,916, filed on Dec. 17, 2021.
(60) Provisional application No. 63/304,086, filed on Jan. 28, 2022.
(60) Provisional application No. 63/369,017, filed on Jul. 21, 2022.

(51) Int. Cl. A61K 31/505; A61K 45/06; A61P 37/08

(52) U.S. Cl. CPC A61K 31/505; A61K 45/06; A61P 37/08

(57) ABSTRACT

The present disclosure relates to methods of treating and/or preventing IgE driven allergic reaction to one or more allergens, preferably food allergens, in subjects having such disease or condition, comprising administering a therapeuticallly effective dose of LOU064.

### TECHNICAL FIELD

The present invention concerns BTK inhibitors, e.g. LOU064 or a pharmaceutically acceptable salt or polymorph thereof for use in the effective suppression of IgE driven allergic reaction to one or more allergens.

### BACKGROUND OF THE DISCLOSURE

Food allergy affects millions of people of all ages in all nations, and a rapidly rising prevalence suggests it is an emerging population health priority (Warren et al 2020). The underlying pathogenesis of food allergy involves an immunologic mechanism in which allergen-specific IgE is synthesized in response to allergen exposure and binds to high affinity receptors for IgE (FcERI receptors) via its Fc region on the surface membranes of mast cells and basophils. (Sampson et al 2006). Food allergies affect almost 10% of people worldwide, with consistent epidemiology trends observed across North America, Europe, Asia and Australia; the most common allergens are: peanut, tree nuts, seafood, egg, milk, wheat, soy and seeds (Warren et al 2020, Sicherer and Sampson 2017). Allergy to peanut, tree nuts and seafood are usually lifelong (Jones and Burks 2017, Sicherer and Sampson 2017). Additionally, between one-third to one-half of food allergic patients-including adults—are likely to be allergic to more than one food (Gupta et al 2011, Gupta et al 2019). Anaphylaxis is a possibly life-threatening systemic allergic reaction to substances such as foods, drugs, or stinging insect venoms. Despite efforts to mitigate allergies to these substances with immunotherapy, the prevalence of anaphylaxis is increasing (Lieberman P. et al., Ann Allergy Asthma Immunol. 2006; 97 (5): 596-602). Unfortunately, it is often fatal, even with prompt and comprehensive medical treatment (J. Allergy Clin. Immunol. 2020; 145 (4): 1082). There are no known therapies that can prevent IgE-mediated anaphylaxis.

The IgE pathway is a central pathogenic player in most allergic disorders, including food allergy, drug allergy, allergic rhinitis, asthma, and chronic urticaria. When allergen-specific IgE binds allergen, cross-linking high-affinity receptors (FcERI) on the surface of mast cells and basophils, the activated signaling cascade causes the release of numerous allergic mediators including histamine, prostaglandins, leukotrienes, and cytokines that are responsible for inducing signs and symptoms.

In the past, the treatment of allergic disorders has largely depended on the blockade of specific mediators (e.g., with antihistamines or leukotriene receptor antagonists) and/or broad immune suppression with corticosteroids.

Currently there are no effective treatments capable of preventing anaphylaxis, and the mainstay of treatment is prevention by avoidance of triggers. However, strict avoidance is not always possible, especially in the case of food allergy, so standard of care consists of early recognition of signs and symptoms and the use of injected intramuscular epinephrine after a reaction has occurred. In contrast, there are situations in which a patient is intentionally exposed to an allergen such as during skin testing, allergen immunotherapy (IT), and drug desensitizations, all procedures that carry a risk of a serious systemic reaction. Desensitizations to allergens, such as e.g. food allergens or medications, are expensive and labor-intensive, as these typically require inpatient admission due to the risk of potentially life-threatening reaction.

There have been several immunotherapies tested for food allergy such as oral immunotherapy (OIT), sublingual immunotherapy (SLIT), epicutaneous immunotherapy (EPIT), and OIT combined with anti-IgE monoclonal antibodies (omalizumab). However, efficacy and safety have only been demonstrated in one Phase III trial for peanut allergies. Additionally, there have only been three randomised, controlled studies of omalizumab-OIT combination and these were low-powered, single-centre trials; therefore, evidence levels were low in these trials. Studies which included long-term follow-up observation are rare and therefore, clinical tolerance is not well-defined and remains unknown (Shoichiro T et al. EMJ. 2019; 4 [4]: 63-70).

Today, there is only one approved oral immunotherapy (OIT) for peanut allergic patients (Palforzia™).

BTK is an essential kinase for signaling through FcERI in human mast cells and basophils. Because it is also crucial for B cell maturation, BTK has been pharmacologically targeted for the treatment of B cell malignancies. There are currently three FDA-approved BTK inhibitors (BTKis) in the USA. Ibrutinib (brand name Imbruvica®; Pharmacyclics, and AbbVie), acalabrutinib (Calquence®; Acerta and AstraZeneca), and zanubrutinib (Brukinsa®; BeiGene) are all oral, covalent small molecule inhibitors of BTK.

Furthermore, several BTK inhibitors (BTKis) are in clinical trials for the treatment of auto-immune diseases such as rheumatoid arthritis (RA), multiple sclerosis (MS), and systemic lupus erythematosus (SLE). For example, acalabrutinib and zanubrutinib as well as the novel compounds ONO-4059 (tirabrutinib), HM71224 (poseltinib) and ABBV-105 (upadacitinib) are currently being tested for their efficacy in B cell malignancies and/or autoimmune diseases such RA, Sjogren's Syndrome (SjS) and SLE. Additionally, evobrutinib, tolebrutinib and fenebrutinib have entered phase III studies in MS patients, orelabrutinib is tested in a phase II study and BIIB091 was tested in a phase I study for efficacy in the treatment of MS.

Studies have also demonstrated the ability of ibrutinib to reduce or eliminate skin prick test reactivity to aeroallergens and foods in allergic subjects (Dispenza et al. J. Allergy Clin Immunol. 2018; 141 (5): 1914-1916.e7) and that the skin tests had returned to baseline 1 week after cessation of ibrutinib suggesting a short duration of efficacy (2021). However, it is unknown whether inhibitory effects seen in the skin prick test will translate into inhibitory effect in organs other than the skin. The penetrance of BTKis into various tissues where mast cells reside is unknown. Even though it has been demonstrated that ibrutinib has a rapid efficacy in preventing IgE-mediated activation of circulating basophils, it is unknown whether BTKis would fully inhibit IgE-mediated activation of tissue-resident mast cells in multiple organs such as the lung, skin, and gastrointestinal tract.

Despite suggestion that BTK inhibitors may be used for the prevention and/or treatment of allergic reaction to food, BTK inhibitors have not yet been shown to suppress clinically relevant allergic reactions and/or prevent anaphylaxis in humans. No clinical efficacy data of BTK inhibitors in relation to food allergy have been reported up to date, even though acalabrutinib has now entered a phase II trial in prevention of allergic reactions to peanut allergen in peanut allergic adults.

Furthermore, due to the lack of selectivity of some earlier developed BTK inhibitors (e.g. acalabrutinib, ibrutinib), those BTK inhibitors may not be suitable for non-malignant indications, especially for the treatment of indications which requires long term/chronic and safe use, and/or in a pediatric or adolescent population. The most common side effects of the currently approved BTK inhibitors include nausea, diarrhea, rash, infection, cytopenias, bleeding and cardiac arrhythmias. Particularly, the long-term toxicity profile of ibrutinib, a first-in-class inhibitor, is well characterized and includes a clinically significant incidence of cardiac arrhythmias, bleeding, infection, diarrhea, arthralgias, and hypertension. Acalabrutinib, the initial second-generation BTKi to earn approval from the US Food and Drug Administration, demonstrates improved kinase selectivity for BTK, with commonly observed adverse reactions including infection, headache, and diarrhea (Hematology Am Soc Hematol Educ Program. 2020 Dec. 4; 2020 (1): 336-345).

Long term safety data is not available for BTK inhibitor currently in development but available safety data for fenebrutinib may suggest that the biggest obstacle that BTK inhibitors may face is whether side effects during chronic use will be acceptable. For example, a major event noted for fenebrutinib's phase IIa trial was transient grade 3 elevations of ALT and/or AST in 8.3% and 6.3% of the subjects in the higher dose treatment arms. Some of the side effects already observed for some BTKis may limit their use for treating or preventing non-malignant indications, especially if would requrie a long-term administration. Additionally none of the FDA-approved BTK inhibitors have been approved in children and adolescents. Therefore, until safety data in children is available it is unknown whether BTK inhibitors will be a viable option for use in treating or preventing IgE driven allergic reaction in children.

Due to rising prevalence (including allergy to multiple foods), currently limited therapeutic options, potentially life-threatening consequences, the inability of food avoidance alone to offer protection and lifelong disease burden in many, there is a significant medical need to develop novel and safe therapies for food allergy.

### BRIEF DESCRIPTION OF THE FIGURES

FIG. 1: Passive cutaneous analphylaxis (PCA) in mice: LOU064 inhibits skin edema

FIG. 2: BTK occupancy in spleen of BALB/c mice 2.5 h after dosing

FIG. 3: Reverse passive arthus (RPA) reaction in mouse skin: LOU064 inhibits skin swelling

FIG. 4: BTK occupancy in spleen of C57B16 mice 5 h after dosing

FIG. 5: Time dependent pharmacologic effect of LOU064 on skin swelling

FIG. 6: Comparison of time-dependent of BTK occupancy in spleen and lung to pharmacologic inhibition of skin swelling after a single dose of 30 mg/kg LOU064

FIG. 7: Effect on total number of cells in brochoalveolar lavage fluid (BALF) following treatment with LOU064 (10 mg/Kg and 30 mg/kg, p.o., b.i.d), or vehicle (10 ml/Kg, p.o., b.i.d.) treatment in sensitized mice challenged with 1% w/v OVA or saline (aerosol., days 21-24) and 16 h with PBS and 16 h with PBS (i.t. Group 1 and 2) or polyinosine-polycytidylic acid (Poly I: C 100 μg/animal, i.t.). BALF was collected 24 h after OVA challenge.

FIG. 8: Effect on the number of eosinophils in brochoalveolar lavage fluid (BALF) following treatment with LOU064 (10 mg/Kg and 30 mg/kg, p.o., b.i.d), or vehicle (10 mL/Kg, p.o., b.i.d.) treatment in sensitized mice challenged with 1% w/v OVA or saline (aerosol., days 21-24) and 16 h with PBS and 16 h with PBS (i.t. Group 1 and 2) or polyinosine-polycytidylic acid (Poly I: C 100 μg/animal, i.t.). BALF was collected 24 h after OVA challenge.

FIG. 9: Effect on the number of neutrophils in brochoalveolar lavage fluid (BALF) following treatment with LOU064 (10 mg/kg and 30 mg/kg, p.o., b.i.d), or vehicle (10 ml/Kg, p.o., b.i.d.) treatment in sensitized mice challenged with 1% w/v OVA or saline (aerosol., days 21-24) and 16 h with PBS and 16 h with PBS (i.t. Group 1 and 2) or polyinosine-polycytidylic acid (Poly I: C 100 μg/animal, i.t.). BALF was collected 24 h after OVA challenge.

FIG. 10: Effect on the number of macrophages in brochoalveolar lavage fluid (BALF) following treatment with LOU064 (10 mg/kg and 30 mg/kg, p.o., b.i.d), or vehicle (10 ml/Kg, p.o., b.i.d.) treatment in sensitized mice challenged with 1% w/v OVA or saline (aerosol., days 21-24) and 16 h with PBS and 16 h with PBS (i.t. Group 1 and 2) or polyinosine-polycytidylic acid (Poly I: C 100 μg/animal, i.t.). BALF was collected 24 h after OVA challenge.

FIG. 11: Effect on the number of lymphocytes in brochoalveolar lavage fluid (BALF) following treatment with LOU064 (10 mg/Kg and 30 mg/kg, p.o., b.i.d), or vehicle (10 ml/Kg, p.o., b.i.d.) treatment in sensitized mice challenged with 1% w/v OVA or saline (aerosol., days 21-24) and 16 h with PBS and 16 h with PBS (i.t. Group 1 and 2) or polyinosine-polycytidylic acid (Poly I: C 100 μg/animal, i.t.). BALF was collected 24 h after OVA challenge.

FIG. 12: Preferred particle size distribution of nanosized LOU064.

FIG. 13: Simulation of Spleen BTK occupancy at steady state.

FIG. 14: (A) Trough over 24 hours of BTK Occupancy at steady state. Graph showing the median prediction as point and the vertical lines showing the 95% prediction interval. (B) Average over 24 hours of BTK Occupancy at steady state. Graph showing the median prediction as point and the vertical lines showing the 95% prediction interval.

### SUMMARY OF THE DISCLOSURE

The problem underlying the present invention is to provide a safe and efficacious treatment or a safe and effective prevention of IgE driven allergic reaction, e.g. food, drug allergy or venom allergic reaction, particularly IgE driven allergic reaction to one or more allergens, e.g. food allergens, and more particularly e.g. IgE driven anaphylactic reaction. In particular, it is an object of the present invention to provide a safe and efficacious long-term treatment or prevention of IgE driven food allergic reaction, e.g. anaphylaxis reaction.

Accordingly, the invention relates to BTK inhibitor, e.g. reversible or irreversible BTK inhibitor, for use in the treatment and/or prevention of IgE driven allergic reaction, e.g. food, drug, or venom allergic reaction, more particularly food allergic reaction. Particularly the invention relates to a selective BTK inhibitor which is safe and suitable for long term use (i.e. chronic use) in the treatment and/or prevention of IgE driven allergic reaction, e.g. food, drug or venom allergic reactions, more particularly food allergic reaction. Particularly useful for the invention is a BTK inhibitor which is selective against other structurally similar Tec family kinases such as BMX, ITK and TXK. More particularly useful for the invention is a BTK inhibitor which is selective for BTK over tec (e.g. a BTK/Tec selectivity of at least 10 fold, at least 20 fold, at least 30 fold).

It is another object of the invention to provide an improved treatment and/or prevention of IgE driven allergic reaction, e.g. food, drug, or venom allergic reactions, more particularly food allergic reaction. One aspect of the invention is to provide an improved prevention of IgE driven anaphylaxis, e.g. induced by one or a mixture of food allergens. For example, it is an object of the invention to provide a treatment or prevention of IgE driven allergies which is more effective and/or safer than treatment with other BTK inhibitors (e.g. less selective BTK inhibitors), particularly a more effective and/or safe treatment than treatment with acalabrutinib, or ibrutinib. It is also an object of this invention to provide a treatment and/or prevention of IgE driven allergic reaction, particularly food allergic reaction, which is more effective, safer and/or more convenient than treatment with anti-IgE monoclonal therapies. Particularly, it is the object of this invention to provide a treatment and/or prevention of IgE driven allergic reaction which has a faster onset of action (e.g. less than 4 weeks, less than 2 weeks or less than one week) as compared to anti-IgE monoclonal therapies which takes 8-12 weeks for an onset of action and to provide a therapy which is more conveniently administered at home and does not require the use of a healthcare setting.

Accordingly, disclosed herein are methods of preventing or treating IgE driven allergic reaction, e.g. food, drug, or venom allergic reaction, e.g. anaphylactic reaction, more particularly methods of preventing or treating IgE driven allergic reaction to one or more food allergens (e.g. peanut, tree nut, milk, wheat, soy, egg, sesame, fish and shelfish or mixture thereof) comprising administering a therapeutically effective dose of LOU064 to a subject in need thereof.

Another aspect of the invention relates to the use of an irreversible BTK inhibitor, e.g. LOU064 or a pharmaceutically acceptable salt thereof, for treating or preventing IgE driven food allergic reaction to one or more allergens (e.g. food allergens such as peanut, tree nut, milk, wheat, soy, egg, sesame, fish and shelfish or mixture thereof), preferably preventing IgE driven food allergic reaction to one or more allergens.

A further subject of the present invention is a method for the manufacture of a medicament for use in the treatments described above.

Further aspects, advantageous features and preferred embodiments of the present invention which are summarized in the following embodiments E1-E39, respectively alone or in combination, contribute to solving the object of the invention:
E1. A method of treating or preventing an IgE driven allergic reaction to one or more allergens (e.g. drug, venoms or food allergens), in a subject in need thereof, comprising administering a therapeutically effective dose of a selective BTK inhibitor, e.g. LOU064, to a patient.
E2. The method according to E1, wherein one or more allergens comprises or is food allergens.
E3. The method according to E2, wherein food allergen is selected from peanut, tree nut, milk, wheat, egg, soy, sesame, fish and shelfish, particularly peanut.
E4. The method according to E1, E2 or E3, wherein the therapeutically effective dose of LOU064 is from about 20 mg to about 200 mg daily.
E5. The method according to E4, wherein the therapeutically effective dose of LOU064 is from about 10 mg twice daily to about 100 mg twice daily
E6. The method according to E5, wherein the therapeutically effective dose of LOU064 is about 10 mg twice daily.
E7. The method according to E5, wherein the therapeutically effective dose of LOU064 is about 25 mg twice daily.
E8. The method according to E5, wherein the therapeutically effective dose of LOU064 is about 100 mg twice daily.
E9. The method according to any one of E1-E8, wherein LOU064 is administered for a short term, e.g. less than 6 months, preferably less than 3 months or less than 1 month.
E10. The method according to E9, wherein LOU064 is administered during up to 18 weeks, e.g. during 4, 10, 12, 16 or 18 weeks.
E11. The method according to any one of E1 to E8, wherein LOU064 is administered for a long term, e.g. more than 6 months, e.g. one year, preferably more than a year.
E12. The method according to any one of E1-E11, wherein LOU064 is administered as a monotherapy.
E13. The method according to any one of E1-E12, wherein LOU064 is not administered concomitantly with a strong inhibitor of CYP3A, e.g. a strong inhibitor of CYP3A4.
E14. The method according to any one of E1-E11 and E13, wherein LOU064 is co-administered with a therapeutic agent.
E15. The method according to E14, wherein LOU064 is co-administered with a corticosteroid and/or an immunosuppressor (e.g. inhaled corticosteroid), a leukotriene receptor antagonist (LTRA), a short-acting beta agonist (SABA) or a long-acting beta agonist (LABA).
E16. The method according to any one of claims E1-E11 and E13, wherein LOU064 is co-administered with an oral immunotherapy (OIT), sublingual immunotherapy (SLIT), epicutaneous immunotherapy (EPIT), preferably an OIT.
E17. The method according to E16 wherein LOU064 is an adjunct to oral immunotherapy (OIT), sublingual immunotherapy (SLIT), epicutaneous immunotherapy (EPIT), preferably an OIT.
E18. The method according to E16 or E17 wherein the oral immunotherapy is a peanut protein (e.g. Palforzia).
E19. The method according to any one of E16 to E18, wherein LOU064 is administered starting at least 2 days (e.g. at least 2-14 days) prior to administration of the oral immunotherapy.
E20. The method according to E19 wherein LOU064 is administered during the escalation phase of the immunotherapy treatment.
E21. The method according to any one of E1-E20 wherein the method is prevention of an IgE driven allergic reaction.
E22. A method according to any one of E1 to E21 for preventing anaphylaxis after accidental exposure to any allergens or mixtures thereof (e.g. food allergen).
E23. The method according to E21 or E22 wherein LOU064 achieves maximal prevention after a minimum of 2 days (e.g. after 2-14 days, preferably after 2-7 days) of treatment.
E24. The method according to any one of E1-E23 wherein the patient is selected according to one or more of the following criteria:
   (a) Male and female patients, 2 years of age or older (e.g. 2 to 5 years of age, or 6 to 11 years of age, 12-17 years of age or 18-55 years of age);
   (b) Documented medical history of allergy to foods, including but not limited to peanuts, tree nuts, wheat, egg, milk, soy, fish and shellfish;
   (c) Positive allergen-specific IgE (e.g. peanut sigE ≥6 kUA/L at Screening); and
   (d) Skin prick test positive for allergen to which patient is allergic (e.g. defined as the average diameter (longest diameter and mid-point orthogonal diameter) ≥4 mm wheal compared to the negative control).
E25. The method according to any of E1-E24, wherein the patient is an adult patient (18 years of age and above) or an adolescent (12-17 years of age).
E26. The method according to any of E1-E24, wherein the patient is a child of age 2 to 11, e.g. age 2 to 5 or age 6 to 11.
E27. The method according to any one of the E1-E26 wherein at least one of the following applies:
   a. At least 90% of the treated patients do not exhibit an allergic reaction upon food challenge with 600 mg of peanut protein
   b. At least 90% of the treated patients do not exhibit an allergic reaction upon food challenge with 1000 mg of peanut protein
   c. At least 80% of the treated patients do not exhibit an allergic reaction upon food challenge with 3000 mg of peanut protein;
after up to 4 weeks of treatment with LOU064 (e.g. after 1 week, or after 2 weeks, or after 3 weeks or after 4 weeks).
E28. The method according to any one of the E1-E27 wherein the patient achieves a reduction from baseline in the total domain score FAQLQ of 0.45-0.5.
E29. The method according to any one of E1-E28 wherein the patient achieves a reduction from baseline in the total domain score FAIM.
E30. The method according to any one of E1-E29, wherein in a double-blind placebo-controlled food challenge with 600 mg of an allergen (e.g peanut allergen), the observed difference in the responder rate between the treated and non-treated patients is superior to 35%, wherein the responder rate is defined as no more than a mild response to 600 mg oral food challenge.
E31. The method according to any one of the E1-E30, wherein by week 12 or by week 24 of treatment the levels of alanine aminotransferase (ALT), aspartate aminotransferase (AST), and lipase do not change by more than 10% as compared to the baseline level at the start of therapy.
E32. The method according to any one of E1-E31, wherein LOU064 is administered in the form of a suitable oral pharmaceutical formulation comprising nanosized particles of LOU064.
E33. The method according to any one of E1-E32, wherein LOU064 is administered in the form of a suitable oral pharmaceutical formulation comprising nanosized particles of LOU064 having a mean particle size as measured by PCS of between about 50 nm to about 750 nm.
E34. The method according to any one of E1-E33, wherein LOU064 is administered in the form of a suitable oral pharmaceutical formulation comprising LOU064 and binder at a weight ratio of about 2:1.
E35. The method according to any one of E1-E34, wherein LOU064 is administered in the form of a suitable oral pharmaceutical formulation comprising LOU064, binder and surfactant at a weight ratio of about 2:1:0.08.
E36. The method according to any one of E1 to E33, wherein LOU064 is administered in the form of a suitable oral pharmaceutical formulation comprising LOU064 and binder at a weight ratio of about 1:1.
E37. The method according to any of E1 to E33 and E36, wherein LOU064 is administered in the form of a suitable oral pharmaceutical formulation comprising LOU064, binder and surfactant at a weight ratio of about 1:1:0.05.
E38. The method according to any one of E1-E37, wherein LOU064 is administered in the form of a suitable oral pharmaceutical formulation comprising LOU064, polyvinylpyrrolidone-vinyl acetate copolymer as a binder and sodium lauryl sulfate as a surfactant.
E39. The method according to any of E1-E38 wherein LOU064 is a crystalline form of the anhydrous free base characterized by an x-ray powder diffraction pattern comprising one or more representative peaks in terms of 20 selected from the group consisting of 7.8±0.2° 20, 9.2±0.2° 20, 12.0±0.2° 20, 13.6±0.2° 20, 15.6±0.2° 20, 16.0±0.2° 20, 17.8±0.2° 20, 18.3 ±0.2° 20, 18.7±0.2° 20, 19.2±0.2° 20, 19.9±0.2° 20, 22.1±0.2° 20, 23.4±0.2° 20, 23.9±0.2° 20, 24.8±0.2° 20, 25.2±0.2° 20, 25.5±0.2° 20, 27.2±0.2° 20, and 29.6±0.2° 20, when measured at a temperature of about 25° C. and an x-ray wavelength, λ, of 1.5405 Å.

### DETAILED DESCRIPTION OF THE DISCLOSURE

### Definitions

As used herein, Bruton's tyrosine kinase (BTK) is a cytoplasmic tyrosine kinase and member of the TEC kinase family. BTK is expressed in selected cells of the adaptive and innate immune system including B cells, macrophages, mast cells/basophils and thrombocytes. BTK is indispensable for signaling through the Fc epsilon receptor (FcER1 for IgE) and the activating Fc gamma receptors (FcγR for IgG), as well as the B cell antigen receptor (BCR) and BTK inhibitors. BTK inhibitors like ibrutinib are approved for the treatment of B cell malignancies (Hendriks et al 2014). Recently, it has been demonstrated that inhibition of BTK leads to inhibition of mast cell and basophil activation/degranulation in vitro and to reduced wheal sizes in skin prick tests with patients suffering from IgE-mediated allergies (Smiljkovic et al 2017; Regan et al 2017; Dispenza et al 2018). Therefore, inhibition of BTK is an attractive therapeutic concept to treat various autoimmune and chronic inflammatory diseases, including rheumatoid arthritis, multiple sclerosis, systemic lupus erythematosus, chronic urticaria, atopic dermatitis, asthma, and primary Sjogren's Syndrome (Tan et al 2013; Whang and Chang 2014). Examples of BTK inhibitors include non-covalent, reversible BTK inhibitors such as fenebrutinib as well as covalent, irreversible inhibitors of BTK such as evobrutinib, tolebrutinib, rilzabrutinib, tirabrutinib, branebrutinib, orelabrutinib and remibrutinib (LOU064).

As used herein, IgE refers to Immunoglobulin E.

The term “comprising” encompasses “including” as well as “consisting,” e.g., a composition “comprising” X may consist exclusively of X or may include something additional, e.g., X+Y.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salt”,” or “salts” refers to an acid addition salt of a compound of the invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable. In many cases, the compound of the present invention is capable of forming acid salt by virtue of the presence of amino group thereto. Example of salts are those disclosed in WO2020/234779 and WO2015/079417 which are hereby incorporated by reference.

As used herein, the term “polymorph” refers to a crystalline form having the same chemical composition but different spatial arrangements of the molecules, atoms, and/or ions forming a crystal. Crystalline forms of LOU064 are disclosed in WO2020/234779 which is hereby incorporated by reference.

The term “administering” in relation to a compound, e.g., LOU064, is used to refer to delivery of that compound to a subject by any route, preferably oral administration.

As used herein, the term “a therapeutically effective amount or dose” of a compound (i.e. compound of Formula (I) or a pharmaceutically acceptable salt or polymorph thereof of the present disclosure refers to an amount of the compound of the present disclosure that will elicit the biological or medical response of a subject (patient of subject), for example, reduction or inhibition of an enzyme or a protein activity, or ameliorate symptoms, alleviate conditions, slow or delay disease progression, or prevent a disease, etc. The therapeutically effective dosage of a compound, the pharmaceutical composition, or the combinations thereof, is dependent on the species of the patient, the body weight, age, sex, and individual condition, the disorder or disease or the severity thereof being treated. Frequency of dosage varies depending on the compound used and the particular condition to be treated or prevented.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “treatment” or “treat” is herein defined as the application or administration of a BTK inhibitor, e.g. LOU064 or a pharmaceutically acceptable salt thereof, or a pharmaceutical composition comprising LOU064 or a pharmaceutically acceptable salt thereof, to a subject (preferably human) or to an isolated tissue or cell line from a subject, where the subject has a particular disease (e.g., IgE driven food allergies), a symptom associated with the disease (e.g., IgE driven food allergic reaction), or a predisposition towards development of the disease (if applicable), where the purpose is to cure (if applicable), reduce the severity of, alleviate, ameliorate one or more symptoms of the disease, improve the disease, reduce or improve any associated symptoms of the disease or the predisposition toward the development of the disease. The term “treatment” or “treat” includes treating a subject suspected to have the disease as well as subjects who are ill or who have been diagnosed as suffering from the disease or medical condition.

As used herein, the term “prevention”, “prevent”, “preventing” of a disease or disorder (IgE driven food allergies) refers to the prophylactic treatment of the disease or disorder; or delaying and or suppressing the onset or progression of the disease or disorder (e.g. suppressing the IgE driven food allergic reaction). The term “prevention” refers to decreasing sensitivity to an allergen in a patient subject to an allergic reaction to said allergen (i.e. increasing the threshold of sensitivity to an allergen as assessed by an oral food challenge). The term includes for example, attenuating a food allergic reaction(s) upon oral exposure to a known allergens (e.g. food allergen) by a minimum of 80% from the baseline (e.g. 85%, 90% from baseline). The term also includes protection against a potentially life-threatening IgE-driven allergic reaction and more particularly IgE-driven anaphylaxis.

As used herein, the term “anaphylaxis” is defined according to World Allergy Organization Guidance 2020 as as a severe, life-threatening systemic hypersensitivity reaction characterized by being rapid in onset with potentially life-threatening airway, breathing, or circulatory problems and is usually, although not always, associated with skin and mucosal changes. A large variety of molecules can induce anaphylaxis. These are most frequently proteins, which induce anaphylaxis in an IgE-dependent manner or molecules, which directly activate mast cells. An IgE-mediated anaphylaxis is triggered by the interaction of an allergen (usually a protein) interacting with the allergen-specific IgE/high-affinity receptor (FcERI) complex expressed on effector cells, predominantly mast cells and basophils.

As used herein, the phrase “population of subjects” is used to mean a group of subjects.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In a preferred embodiment, the subject is a human. The term “subject” is used interchangeably with “patient” when it refers to human.

As used herein, a subject is “in need of” a treatment or prevention if such subject would benefit biologically, medically or in quality of life from such treatment.

In some embodiments of the disclosed methods, the BTK inhibitor, e.g. LOU064 or a pharmaceutically acceptable salt thereof, is used to treat a population of subjects with IgE driven food allergic reaction to one or more allergens.

As herein defined, the term “IgE driven allergy (or allergic reaction) to one or more allergens” or “IgE mediated allergy (or allergic reaction)” are used interchangeably and refer to e.g. drug allergy, venom allergy or food allergy (or allergic reaction), preferably allergic reactions to food allergens such as peanut, tree nut, milk, wheat, soy, egg, sesame, fish and shelfish or mixture thereof. In some embodiments, food allergy also refers to an indication for the prevention of allergic reactions, including anaphylaxis, following accidental exposure to food allergens, e.g. in adult and paediatric patients 2 years of age and older, with a confirmed or suspected diagnosis of IgE driven food allergy to one or more allergens. The term, “IgE driven food allergic reaction” is an adverse reaction to foods and is driven by uncontrolled type-2 immune responses.

In some embodiments, drug allergy refers to an indication for the prevention of allergic reactions, including anaphylaxis, following exposure to the drug allergen(s), e.g. in adult and paediatric patients 2 years of age and older, e.g. with a confirmed or suspected diagnosis of IgE driven allergy to one or more allergens.

The IgE-mediated reactions are usually divided into immediate-onset reactions (arising up to 2 hours from the food ingestion) and immediate plus late-phase (in which the immediate onset symptoms are followed by prolonged or ongoing symptoms). IgE mediated drug or food related reactions includes skin manifestations (e.g. angiodema, acute urticaria); gastrointestinal tract reactions (symptoms can include lip, tongue and palatal pruritus and swelling, laryngeal oedema, nausea, abdominal cramping, vomiting and diarrhoea); respiratory reactions (e.g. Allergic rhinoconjunctivitis and bronchoconstriction); systemic reactions (anaphylaxis which is a rapidly progressive, multiple organ system reaction which is typically combined with gastrointestinal (GI) or respiratory symptoms and include cardiovascular collapse).

Non-responders to therapy using a BTK inhibitor, e.g. LOU064 or a pharmaceutically acceptable salt thereof, are defined as subjects who failed to achieve at least 80% improvement of their baseline or had an exacerbation of their symptoms. Responders to therapy using LOU064 or a pharmaceutical acceptable salt thereof are defined as those subjects who achieved a minimum of 80% improvement of baseline.

The term “oral immunotherapy” or “OIT” is a therapy in which patient with allergies consume gradually over many months increasing quantities of an allergen (e.g. a food allergen) to which there are allergic in order to elicit safer immune responses to said allergen and induced some level of desensitization. OIT therapy consists of 2 phases: 1. Escalation phase which consists of slowly increasing the amount of allergen—this phase can take many months; and 2. A maintenance phase where the patient remains on the same dose. Unfortunately the escalation phase presents risk of severe allergic reactions including anaphylaxis. Patients who can reach maintenance dose are generally protected against reactions due to accidental exposures as long as they are taking OIT.

The term “sublingual immunotherapy” or SLIT is an another form of allergy immunotherapy which involves administering the allergens under the tongue generally on a daily basis. Currently, the only forms of SLIT approved by the FDA are tablets for ragweed, northern pasture grasses like timothy and dust mites. The tablets are placed under the tongue for one to two minutes and then swallowed as they dissolve. The process is repeated from three days a week to as often as daily. The tablets will increase tolerance to the pollen and reduce symptoms over time. For continued effectiveness, treatment may be needed for three years or longer. Allergy tablets are currently available for ragweed and grass pollen only.

The term “epicutaneous immunotherapy” or EPIT is another form or allergen immunotherapy which consists in delivering the allegen via repeated applications to the skin. EPIT uses patches and aim to develop sustained sensitization or tolerance by continuous (and constant) allergen exposure to the skin.

As used herein, “selecting” and “selected” in reference to a patient is used to mean that a particular patient is specifically chosen from a larger group of patients on the basis of (due to) the particular patient having a predetermined criteria. Similarly, “selectively treating” or “selectively preventing” refers to providing treatment or prevention to a patient having a particular disease (e.g. patients being known or suspected to have allergic reactions to certain food allergens), where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By “selecting”, “selectively treating” and “selectively administering”, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient was selected for treatment based on having known allergic reactions to exposure to food allergens.

The term “pharmaceutical combination” as used herein means a product that results from the use or mixing or combining of more than one active ingredient. It should be understood that pharmaceutical combination as used herein includes both fixed and non-fixed combinations of the active ingredients. The term “fixed combination” means that the active ingredients, e.g., a compound of formula (I) or a pharmaceutically acceptable salt or polymorph thereof), and one or more combination partners, are administered to a patient simultaneously as a single entity or dosage form. The term in such case refers to a fixed dose combination in one unit dosage form (e.g., capsule, tablet, or sachet). The terms “non-fixed combination” or a “kit of parts” both mean that the active ingredients, e.g., a compound of the present disclosure and one or more combination partners and/or one or more co-agents, are administered or co-administered to a patient independently as separate entities either simultaneously, concurrently or sequentially with no specific time limits wherein such administration provides therapeutically effective levels of the two compounds in the body of the patient, especially where these time intervals allow that the combination partners show a cooperative, e.g., an additive or synergistic effect. The term “non-fixed combination” also applies to cocktail therapy, e.g., the administration of three or more active ingredients. The term “non-fixed combination” thus defines especially administration, use, composition or formulation in the sense that the compounds described herein can be dosed independently of each other, i.e., simultaneously or at different time points. It should be understood that the term “non-fixed combination” also encompasses the use of a single agent, e.g. LOU064 or a pharmaceutically acceptable salt or polymorph thereof, together with one or more fixed combination products with each independent formulation having distinct amounts of the active ingredients contained therein. It should be further understood that the combination products described herein as well as the term “non-fixed combinations” encompasses active ingredients (including the compounds described herein) where the combination partners are administered as entirely separate pharmaceutical dosage forms or as pharmaceutical formulations that are also sold independently of each other. Instructions for the use of the non-fixed combination are or may be provided in the packaging, e.g., leaflet or the like, or in other information that is provided to physicians and/or medical staff. The independent formulations or the parts of the formulation, products, or compositions, can then be administered simultaneously or chronologically staggered, that is the individual parts of the kit of parts can each be administered at different time points and/or with equal or different time intervals for any part of the kit of parts. Particularly, the time intervals for the dosing are chosen such that the effect on the treated disease with the combined use of the parts is larger/greater than the effect obtained by use of only compound of Formula (I) or a pharmaceutically acceptable salt or polymorph thereof; thus the compounds used in pharmaceutical combination described herein are jointly active. The ratio of the total amounts of a compound of formula (I) or a pharmaceutically acceptable salt or polymorph thereof, to a second agent to be administered as a pharmaceutical combination can be varied or adjusted in order to better accommodate the needs of a particular patient sub-population to be treated or the needs of the single patient, which can be due, for example, to age, sex, body weight, etc. of the patients.

The terms “co-administration” or “combined administration” or the like as utilized herein are meant to encompass the administration of one or more compounds described herein together with a selected combination partner to a single subject in need thereof (e.g., a patient or subject), and are intended to include treatment regimens in which the compounds are not necessarily administered by the same route of administration and/or at the same time.

The term “adjunct” or “adjunct therapy” or “adjuvant therapy” is therapy that is given in addition to the primary or initial therapy to maximize effectiveness and improve safety. In the context of this invention, adjunct therapy is the use of a BTK inhibitor (e.g. LOU064) in addition to the primary therapy of oral immunotherapy (OIT). For example, the adjunct therapy (i.e. BTK inhibitor or LOU064) is given before the main treatment (e.g. OIT) and during the escalation phase of the OIT in order to prevent allergic reactions and/or analphylaxis reactions during the escalation phase.

### LOU064

LOU064 (=N-(3-(6-amino-5-(2-(N-methylacrylamido) ethoxy) pyrimidin-4-yl)-5-fluoro-2-methylphenyl)-4-cyclopropyl-2-fluorobenzamide, INN: remibrutinib) is disclosed in WO 2015/079417 A1 as a drug candidate for the selective inhibition of Bruton's tyrosine kinase. This compound is a potent, highly selective, irreversible covalent BTK inhibitor. Due to binding to an inactive conformation of BTK, LOU064 exhibits an exquisite kinase selectivity and, thus, reduces kinase off-target binding and due to covalent inhibition, the compound exhibits a potent and sustained pharmacodynamic effect without the need for extended and high systemic compound exposure (Angst, D. et al., Discovery of LOU064 (Remibrutinib), a Potent and Highly Selective Covalent Inhibitor of Bruton's Tyrosine Kinase, J Med Chem. 2020 May 28;63 (10): 5102-5118).

LOU064 for use in the methods of the invention, is the free base as represented by Formula (I):

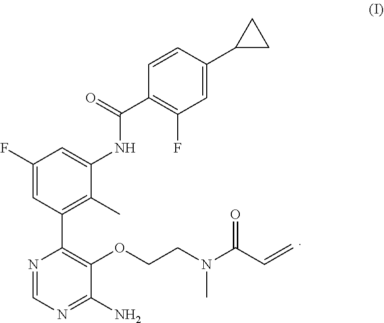 [SMILES: C=CC(=O)N(C)CCOc1c(N)ncnc1-c1cc(F)cc(NC(=O)c2ccc(C3CC3)cc2F)c1C]

In other embodiment, N-(3-(6-amino-5-(2-(N-methylacrylamido) ethoxy) pyrimidin-4-yl)-5-fluoro-2-methylphenyl)-4-cyclopropyl-2-fluorobenzamide is the anhydrous crystalline form A of the free base as disclosed in WO2020/234779 (Example 1), which is thereby incorporated by reference.

LOU064, which has previously been suggested for use in the treatment of chronic spontaneous urticaria (CSU) (WO2020/234782 A1) and Sjoegren's Syndrome (SjS) (WO2020/234781 A1), is currently being tested in clinical studies for SjS (phase 2); CSU and MS (Phase 3).

In WO2020/234782 A1 b.i.d. administration of doses of 10 mg, 25 mg and 100 mg were generally suggested to reach maximal efficacy in CSU.

In a Phase 2b, randomized, double-blind, placebo-controlled trial evaluating the efficacy and safety of remibrutinib over 12 weeks in patients inadequately controlled by H1-antihistamines, with at least moderately active CSU, patients received remibrutinib 10 mg q.d. (once daily), 35 mg q.d., 100 mg q.d., 10 mg b.i.d. (twice daily), 25 mg b.i.d., 100 mg b.i.d., or placebo (1:1:1:1:1:1:1 ratio). The 25 mg b.i.d. regimen was found to be particularly effective.

BTK occupancy in blood and/or tissues has been reported to be a suitable biomarker for selecting doses for clinical studies such as CSU and SjS studies (WO2020/234782 and WO2020/234781).

Furthermore, it has been reported that BTK occupancy and duration of BTK occupancy is different in blood and in various tissues in female rat (WO2020/234781).

BTK occupancy in different tissues is relevant to efficacy and optimum dosage selection in different indications. However, there is no currently agreed complete picture of all tissues that are relevant to the multiple allergic reactions triggered by food allergy and therefore which tissue(s) need to be penetrated for treating or preventing IgE driven food allergic reactions. As reported in rat, the BTK occupancy and BTK occupancy half-life is different in blood and in various tissues.

Furthermore, BTK occupancy half-life is dependent on the turnover rates (ability of the BTK protein within cells to regenerate). Such turnover rates differ in each tissue and are species specific. The BTK occupancy is further dependent on the PK/PD properties of a compound which is also species dependent.

Therefore, clinical response in an oral food challenge cannot be expected using the CSU dose or any doses disclosed for other indications.

### Pharmaceutical Composition for Use in the Methods of the Invention

The BTK inhibitor, i.e., compound of Formula (I), or a pharmaceutically acceptable salt or polymorph thereof, may be used as a pharmaceutical composition when combined with a pharmaceutically acceptable carrier. Such a composition may contain, in addition to the compound of Formula (I) or a pharmaceutically acceptable salt or polymorph thereof, carriers, various diluents, fillers, salts, buffers, stabilizers, solubilizers, and other materials known in the art. The characteristics of the carrier depends on the route of administration. The pharmaceutical compositions for use in the disclosed methods may also contain additional therapeutic agents for treatment of the particular targeted disorder. For example, a pharmaceutical composition may also include anti-inflammatory or anti-itch agents. Such additional factors and/or agents may be included in the pharmaceutical composition to produce a synergistic effect with the compound of Formula (I) or a pharmaceutically acceptable salt or polymorph thereof, or to minimize side effects caused by the compound of Formula (I) or a pharmaceutically acceptable salt or polymorph thereof. In preferred embodiments, the pharmaceutical composition for use in the disclosed methods comprise compound of Formula (I) or a pharmaceutically acceptable salt or polymorph thereof, in a dose of about about 10 mg, about 20 mg, about 25 mg, about 50 mg or about 100 mg, preferably about 25 mg, about 50 mg or about 100 mg.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients, which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Pharmaceutical compositions for use in the disclosed methods may be manufactured in conventional manner. In one embodiment, the pharmaceutical composition is provided for oral administration. For example the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Therefore, pharmaceutical composition for use in the methods of the invention comprises LOU064 and one or more pharmaceutically acceptable carriers, each of which is independently selected from a filler, a lubricant, a binder, a desintegrant and a glidant.

In another embodiment, a suitable pharmaceutical composition, LOU064 may be present in any pharmaceutically acceptable form. It may be preferable that the pharmaceutical composition is in tablet or capsule form. Tablets may be either film coated or enteric coated according to methods known in the art. It may also be preferable to include LOU064 in the pharmaceutical composition/formulation as nanosized or as microsized particles.

If LOU064 is present in the pharmaceutical formulation in the form of nanosized particles, the mean particle size can be less than 1000 nm. Preferably, the mean particle size of LOU064 can be less than 500 nm, more preferably less than 250 nm.

In a preferred embodiment, the mean particle size of LOU064 can be between about 50 nm and about 1000 nm, or between about 50 nm and about 750 nm, or between about 60 nm and about 500 nm, or between about 70 nm and about 350 nm, or between about 100 nm and about 170 nm, More preferably, the mean particle size of LOU064 may be between about 100 nm and about 350 nm, or between about 110 nm and about 200 nm, or between about 120 nm and about 180 nm or between about 120 nm and about 160 nm, preferably the mean particle size of LOU064 can be about 150 nm to about 200 nm.

If LOU064 is present in the pharmaceutical formulation in the form of nanosized particles, oral administration is preferably at a dose of about 50 mg to about 150 mg twice daily, more preferably at a dose of about 100 mg twice daily.

If LOU064 is present in the pharmaceutical formulation in the form of microsized particles, the mean particle size can be 1-5 μm or preferably 1.0-1.5 μm. Preferably, the mean particle size of LOU064 can be 1.1 to 1.3 μm.

If LOU064 is present in the pharmaceutical formulation in the form of microsized particles, oral administration is preferably at a dose of about 100 mg to about 300 mg twice daily, for example at a dose of about 100 mg twice daily.

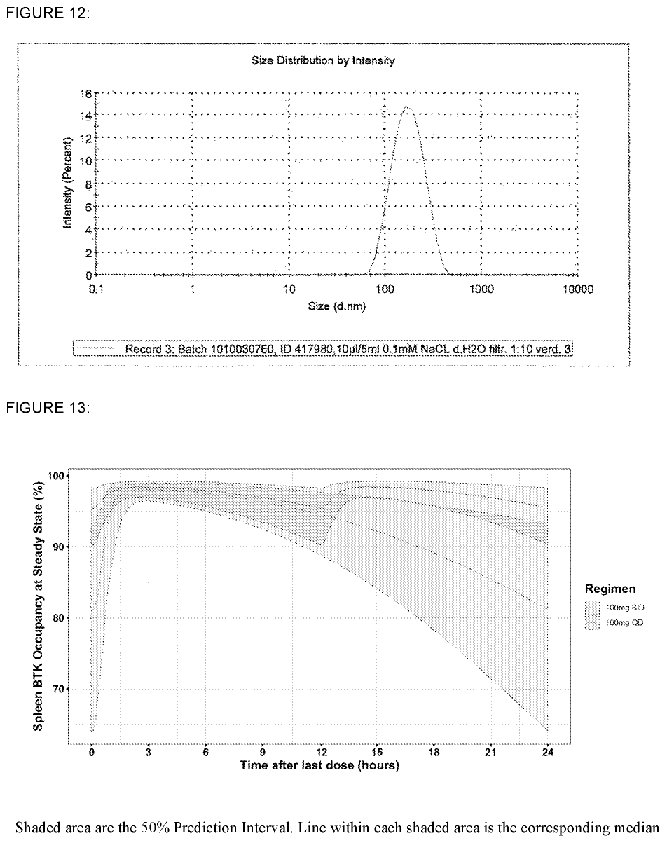

In a preferred embodiment the polydispersity index (PI) is between 0.01 and 0.5, more preferably between 0.1 and 0.2, in particular 0.12-0.14. A preferred particle size distribution is shown in FIG. 12.

The above-mentioned mean particle sizes are intensity weighted. The mean particle size can be determined by means of dynamic light scattering. Preferably, the mean particle size is determined by Photon Correlation Spectroscopy (PCS). In particular, for determining the mean particle size the device “Zetasizer Nano ZS”, Version 7.13 from Malvern Panalytical Ltd., UK can be used.

Preferably, the measurement is carried out as wet dispersion method using 0.1 mM NaCl solution in purified water (1:10), wherein the attenuator index is 2-9, in particular 5. The measurement is preferably carried out at 25° C. Further preferred settings of the measurement systems are as follows:
Cell: Disposable sizing cuvette
Count Rate (kcPs): 315
Duration: 60 sec
Measurement Position (mm): 4.65.

In one embodiment of the invention, a LOU064 composition is formulated in accordance with routine procedures as a pharmaceutical composition adapted for oral administration to human beings. Typically, compositions for oral administration are capsules or tablets.

In one embodiment, a formulation for LOU064 can be formulated according to a formulation disclosed in U.S. application No. 63/141,558 or its family members (e.g. WO2022/162513), herein incorporated by reference.

According to the invention, a suitable pharmaceutical composition for oral administration comprises LOU064 and binder.

Suitable binders include polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, or a mixture thereof. Preferably the binder is polyvinylpyrrolidone-vinyl acetate copolymer.

The weight ratio of LOU064 and binder can be from about 3:1 to about 1:3; e.g. about 3:1, about 2:1, about 1:1, preferably the weight ratio of LOU064 and binder is about 2:1 or about 1:1.

Preferably, a suitable pharmaceutical composition for oral administration comprises LOU064, binder and surfactant.

Suitable surfactants include sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof. Preferably the surfactant is sodium lauryl sulfate.

The weight ratio of LOU064, binder and surfactant is about 2:1:0.5, or about 2:1:0.1, or about 2:1:0.08, or about 2:1:0.05, or about 2:1:0.04, or about 2:1:0.03, or about 2:1:0.02. Preferably, the weight ratio of LOU064, binder and surfactant is about 2:1:0.08 or about 1:1:0.05.

In a particularly preferred embodiment, a suitable pharmaceutical composition for oral administration comprises LOU064, binder and surfactant, wherein the binder is polyvinylpyrrolidone-vinyl acetate copolymer (copovidone) and the surfactant is sodium lauryl sulfate (SLS), and wherein the weight ratio of LOU064, copovidone and SLS is about 2:1:0.08. It is further particularly preferred that LOU064 is present in this pharmaceutical composition in the form of nanosized particles, preferably having a mean particle size as measured by PCS of between about 100 nm and about 200 nm.

### Methods of Treatment or Prevention Using LOU064 or a Pharmaceutically Acceptable Salt Thereof or a Crystalline Form Thereof.

Disclosed methods of, and a BTK inhibitor, e.g an irreversible BTK inhibitor, e.g. compound of Formula (I) (i.e. LOU064) or a pharmaceutically acceptable salt or polymorph thereof, for use in, preventing, treating or modifying the course of IgE driven allergic reactions to one or more allergens, e.g. venom, drug or food, e.g. peanut in a subject in need thereof, comprising herein are administering the subject a therapeutically effective dose of LOU064 or a pharmaceutically acceptable salt thereof. In one aspect of this embodiment, the invention relates to LOU064 or a pharmaceutically acceptable salt thereof for use in, preventing IgE driven food allergic reaction to one or more allergens, e.g. peanut in a subject in need thereof, comprising administering the subject a therapeutically effective dose of LOU064 or a pharmaceutically acceptable salt thereof. LOU064 for use in disclosed methods may be incorporated in a pharmaceutical compositions, as described above, and administered in vivo to treat venom, drug or food allergic patients (i.e. human patients).

For example, IgE driven food allergic reaction to one or more allergens is food allergic reaction, e.g. peanut allergic reaction.

In yet another example, the IgE driven food allergic reaction to one or more allergens disease or disorder refers to peanut, tree nut, milk, wheat, soy, egg, sesame, fish or shelfish allergic reaction.

In another embodiment, the subject is affected by food allergies, e.g. peanut allergy.

The ability of LOU064 to inhibit a defined allergen response was evaluated by mean of the skin prick test (SPT) in healthy atopic subjects in the multiple ascending doses (MAD) study part of the first in human study. SPT with 7 allergens (grass polen mix, birch tree pollen, alder tree pollen, hazelnut tree pollen, house dust mite, cat dander and horse dander) were performed prior to any dosing (baseline) and at different time points after the first dose and after 11 days of once daily dosing. The study demonstrated a dose dependent decrease in wheal size up to 100 mg dose. Beyond 100 mg, a dose dependent trend was not clear, although the highest decrease was observed at the highest tested dose (600 mg q.d.) Accross all tests, participants showed an average of 3 mm decrease in wheal size at doses of greater than or equal to 100 mg qd. (Kaul et al. Clin. Transl. Sci. 2021; 14 (5), pp 1756-1768)

Dose dependent inhibition of FccR1 mediated basophil activation was also demonstrated with LOU064 during first in human study. In single ascending doses (SAD) of LOU064, ex vivo blood basophil activation as measured by CD63 was near completely inhibited (>89%) at doses of 60 mg and reached close to 100% inhibition at higher doses 24 h post dose. Doses greater than or equal to 100 mg demonstrated almost complete inhibition. In contrast, the other basophil activation marker, CD203c+, showed inhibition up to 79.4% at 8h in the 200 mg cohort. In multiple ascending doses (MAD), full inhibition of blood basophil activation measured by CD63 expression was achieved over the entire treatment period (day 1-12) and several days beyond, with a daily dose of LOU064 greater than or equal to 50 mg. Through level inhibition of CD63 on the last day of dosing was 80% with the lowest tested dose of 10 mg. q.d. (once a day); this was stronger with any higher dose. Dose dependent inhibition was observed with CD63 marker reaching greater than 97% trough inhibition with greater than or equal to 100 mg q.d. at the last day of dosing. Inhibition of CD203c+ basophils was highest in the 400 mg q.d. and 200 mg b.i.d at 63% and 67% respectively. (Kaul et al. Clin. Transl. Sci. 2021; 14 (5), pp 1756-1768)

Additionally, we demonstrated in an ovalbumin induced anaphylaxis mice model (example 3) that treatment with LOU064 (10 mg/kg, 30 mg/Kg) significantly reduced numbers of cells involved in lung anaphylaxis (eosiphils, basophils, macrophages and lymphocytes). Based on those results and based on a conversion model between animals and human (Journal of basic and clinical pharmacy, 7 (2), 27-31), the calculated human equivalent dose (HED) for 30 mg/kg corresponds to about 170 mg for a 70 kg person and the calculated human equivalent dose (HED) for the lowest effective dose of 10 mg/kg correspond to about 56 mg for a 70 Kg person (Nair, A. B., & Jacob, S. (2016).

Finally, according to an in house-prediction model of spleen BTK occupancy in human (example 7), a b.i.d. dosing was shown to be more effective than QD dosing at the same dose to achieve higher BTK occupancy (FIG. 13). Accordingly, a dose of LOU064 of 25 mg b.i.d is the minimal human effective dose and a dose of 100 mg b.i.d. is also shown to be effective in human.

The appropriate dosage will vary depending upon, for example, the particular pharmaceutically acceptable salt of LOU064, the particular polymorphic form of LOU064, the host, the mode of administration, the pharmaceutical composition, and the nature and severity of the condition being treated, and on the nature of prior treatments that the subject has undergone.

In a preferred embodiment, LOU064 can be administered irrespective of body weight, sex, age or race. For example, it is preferred that a 35-year-old woman having a body weight of 60 kg receives the same dose as a 50-year old man having a body weight of 90 kg. In particular, body weight, sex, age or race do not have a clinically meaningful effect on the pharmacokinetics of LOU064.

Ultimately, the attending health care provider will decide the amount of LOU064 with which to treat each individual subject. In some embodiments, the attending health care provider may administer low doses of LOU064 and observe the subject's response.

In one embodiment of the present disclosure, LOU064 or a pharmaceutically acceptable salt thereof, is administered orally at a dose of about 20 mg to about 200 mg daily.

In another embodiment, LOU064 or a pharmaceutically acceptable salt thereof, is administered at a dose of about 25 mg daily, or about 50 mg daily, or about 75 mg daily, or about 100 mg daily. In one aspect of this embodiment, LOU064 is administered at a dose of about 25 mg QD (once a day), or about 50 mg QD, or about 75 mg QD, or about 100 mg QD.

In one embodiment of the present disclosure, LOU064 or a pharmaceutically acceptable salt thereof is administered orally at a dose of about 10 mg twice daily to about 100 mg twice daily, e.g. about 10 mg twice daily (B.I.D), about 25 mg B.I.D, about 50 mg B.I.D or about 100 mg B.I.D.

In another embodiment, LOU064 is administered orally at a dose of about 10 mg twice daily.

In another embodiment, LOU064 is administered orally at a dose of about 25 mg twice daily.

In another embodiment, LOU064 is administered orally at a dose of about 50 mg twice daily.

In another embodiment, LOU064 is administered orally at a dose of about 75 mg twice daily.

In another embodiment, LOU064 is administered orally at a dose of about 100 mg twice daily.

The duration of therapy using a pharmaceutical composition of the present disclosure will vary, depending on the severity of the disease or disorder to be treated and the condition and personal response of each individual subject. In some embodiments, the subject is administered LOU064 for a short-term e.g. up to 4 weeks, e.g. up to 6 weeks, e.g. up to 8 weeks, e.g. up to 10 weeks, e.g. up to 12 weeks. LOU064 may for example be administered for example, on a short term basis as defined hereinabove, in order to prevent severe food allergic reactions during a business trip and/or vacation and any other situations where the avoidance of triggers, exposure to allergens is not well controlled.

In some embodiment, LOU064 is administered at least 2 days prior to a potential risk of exposure to allergens (e.g. food allergen). In another embodiment LOU064 is administered within at least one week prior to a potential risk of exposure to allergens (e.g. food allergen), e.g. 1 week before, 2 weeks before, 3 weeks before or 4 weeks before. Advantages of LOU064 over anti-IgE therapies is the fast onset of action for reaching maximum protection/prevention of allergic reactions.

In some other embodiments, the subject is administered LOU064 for a long-term (e.g. LOU064 is used without restriction in total duration for as long as the disease is present justifying its use, e.g. at least 6 months, e.g. at least 10 months, e.g. one year, e.g. for more than 1 year, 2 years, 3 years, 4, years, 10 years. LOU064 or a pharmaceutically acceptable salt thereof might be used up to 2 years, 5 years, 10 years, 15 years, 20 years or for life. In some embodiments, the treatment with LOU064 is a chronic treatment.

In practicing some of the methods of treatment or uses of the present disclosure, a therapeutically effective dose of LOU064 or a pharmaceutically acceptable salt thereof, is administered to a subject, e.g., a mammal (e.g., a human). While it is understood that the disclosed methods provide for treatment or prevention of IgE driven food allergic reactions, using LOU064 or a salt thereof, this does not mean that the therapy is necessarily a monotherapy.

In one aspect of the invention, the BTK inhibitor, e.g. irreversible BTK inhibitor, e.g. LOU064, is administered to the subject as a monotherapy.

In another aspect of the invention, the BTK inhibitor, e.g. irreversible BTK inhibitor, e.g. LOU064, is administered to the subject as a combination therapy.

Indeed, if a subject is selected for treatment with LOU064, then LOU064, may be administered in accordance with the methods of the disclosure either in combination with other agents and therapies for treating the subject affected by a disease or disorder involving IgE, e.g., in combination with at least one additional therapeutic agent, such as e.g., an anti-IgE antibody (e.g. omalizumab or ligelizumab), a corticosteroid or an immunosuppressor, e.g., a systemic corticosteroid or an immunosuppressor.

In some embodiments of the disclosed uses, methods, and kits, the subject has an IgE driven allergic reaction to one or more allergens wherein the allergen is a drug or food, e.g. food, e.g. peanut. In some embodiments, the subject may also have asthma and/or urticaria, e.g., has a disease or disorder selected from asthma, allergic asthma, rhinitis, allergic rhinitis, urticarial and CSU. In some embodiments of the disclosed uses, methods, and kits, the subject has, IgE driven food allergic reaction to one or more allergens (e.g. food allergens such as peanut, tree nut, milk, wheat, soy, egg, sesame, fish and shelfish or mixture thereof)

In other embodiments of the disclosed uses, methods, and kits, the subject has an IgE driven food allergic reaction to one or more allergens wherein the allergen is food, e.g. peanut.

That can be the case for example, when the subject to be treated is allergic, or when the subject is also affected by another disease or disorder selected from asthma, urticaria, and rhinitis, e.g. selected from allergic asthma, CSU, and allergic rhinitis.

When coadministered with one or more additional agent(s), the LOU064 or a pharmaceutically acceptable thereof may be administered either simultaneously with the other agent, or sequentially. If administered sequentially, the attending physician will decide on the appropriate sequence of administering the LOU064 or a pharmaceutically acceptable salt thereof in combination with other agents and the appropriate dosages for co-delivery.

Various therapies may be beneficially combined with the disclosed LOU064, during treatment of the disease or disorder involving IgE, disclosed herein. Such therapies include for example anti IgE antibody (e.g. omalizumab, ligelizumab), corticosteroids (e.g., inhaled or systemic corticosteroids), immunosuppressors, leukotriene receptor anatgonist (LTRA), short-acting beta agonist (SABA) or a long-acting beta agonist (LABA).

In some embodiments of the disclosed uses, methods, and kits, LOU064 or a pharmaceutically acceptable salt thereof, can be prescribed as first treatment or added on to any of the standard of care medications.

In another embodiment, LOU064 or a pharmaceutically acceptable salt, may be administered in accordance with the methods of the disclosure either in combination with other agents and therapies for treating IgE driven food allergic reactions.

In one embodiment, LOU064 or a pharmaceutically acceptable salt thereof is administered together with oral immunotherapy (OIT), sublingual immunotherapy (SLIT), epicutaneous immunotherapy (EPIT), preferably OIT. In one aspect of this embodiment, LOU064 is administered as an adjunct therapy to OIT.

Oral immunotherapy (OIT) is a potential treatment for food allergy, which involves gradually increasing amounts of food allergen given under medical supervision. Such procedure results in desensitizations to the allergens. OIT therapies are expensive and labor-intensive, as these typically require inpatient admission due to the risk of potentially life-threatening reaction.

In one embodiment, the BTK inhibitor, e.g. irreversible BTK inhibitor, e.g. LOU064, when administered in accordance with the methods of the disclosure, reduces the frequency and severity of OIT, SLIT, EPIT, preferably OIT associated side effect (e.g. allergic reactions such as serious systemic reactions, e.g. anaphylaxis).

In another embodiment, the BTK inhibitor, e.g. irreversible BTK inhibitor, e.g. LOU064 when administered in accordance with the methods of the disclosure, shortens the OIT up tritration (i.e. escalation phase of the OIT), for example enabling more patients to successfully complete OIT.

In one embodiment, a BTK inhibitor, e.g. irreversible BTK inhibitor, e.g. LOU064 is administered as an adjunct therapy to an OIT therapy with peanut proteins.

To date the single currently approved OIT product to mitigates allergic reactions in peanut allergic patients in Palforzia™. Therefore, the BTK inhibitor, e.g. irreversible BTK inhibitor, e.g. LOU064 t, may be administered in accordance with the methods of the disclosure as an adjunct to a peanut proteins (e.g. Palforzia™).

In one embodiment, the BTK inhibitor, e.g. irreversible BTK inhibitor, e.g. LOU064 for use according to methods described herein is administered starting at least 2 days, or at least 1 week (7 days), or at least 2 weeks (14 days) prior to administration of the oral immunotherapy. In one aspect of this embodiment, the BTK inhibitor, e.g. irreversible BTK inhibitor, preferably LOU064, for use according to methods described herein is further administered during the length of the escalation phase of the immunotherapy treatment followed by discontinuation of LOU064 at the start of the maintenance phase of the OIT.

### Patient Selection

In one embodiment, the patient is an adult (age 18 and above)

In another embodiment, the patient is an adolescent (age 12-17 years)

In yet another embodiment, the patient is a pediatric patient (age 2-11 years or age 6-11)

In another embodiment of the invention the patient receiving a BTK inhibitor, e.g. irreversible BTK inhibitor, e.g. LOU064 for the treatment or prevention of IgE driven food allergic reactions is selected according to the following criteria:
a) Male and female patients, 2 years of age or older (e.g. 2 to 5 years of age or 6 to 11 years of age, 12-17 years of age or 18-55 years of age);
b) Documented medical history of allergy to foods, including but not limited to peanuts, tree nuts, wheat, egg, milk, soy, fish and shellfish;
c) Positive allergen-specific IgE (e.g. peanut sigE ≥6 kUA/L at Screening);
d) Skin prick test positive for allergen to which patient is allergic (e.g. defined as the average diameter (longest diameter and mid-point orthogonal diameter) ≥4 mm wheal compared to the negative control).

In one aspect of the former embodiment, the patient receiving LOU064 or a pharmaceutically acceptable salt thereof is not a patient with uncontrolled asthma as defined as having one of the following criteria:
FEV1<80% of patient predicted normal value at the first screening visit
One hospitalization for asthma within 12 months prior to the screening visit.

### Efficacy

In one embodiment, the invention relates to a BTK inhibitor, e.g. irreversible BTK inhibitor, e.g. LOU064 for use in all methods described herein wherein the BTK inhibitor, e.g. irreversible BTK inhibitor, e.g. LOU064 achieves prevention at week x of treatment. In one aspect of the this embodiment, prevention is defined as a minimum of 80% reduction from baseline, (e.g. a minumum of 85% reduction or a minimum of 90% reduction from baseline).

In one embodiment, the invention relates to a BTK inhibitor, e.g. irreversible BTK inhibitor, e.g. LOU064 for use in the methods described herein, wherein at least one of the following applies:
At least 90% of the treated patients do not exhibit an allergic reaction upon food challenge with 600 mg of a food allergen (e.g. with 600 mg of peanut protein)
At least 90% of the treated patients do not exhibit an allergic reaction upon food challenge with 1000 mg of food allergen (e.g. with 1000 mg of peanut protein)
At least 80% of the treated patients do not exhibit an allergic reaction upon food challenge with 3000 mg of food allergen (e.g. with 3000 mg of peanut protein)
after up to 4 weeks of treatment with LOU064 (e.g. after 1 week, or after 2 weeks, or after 3 weeks or after 4 weeks)

In another embodiment, the invention relates to LOU064 for use in the methods described herein wherein the patient achieves a reduction from baseline in the total domain score FAQLQ of 0.45 to 0.5.

FALQLQs (food allergy quality of life questionnaires) are disease-specific health-related quality of life (HRQOL) questionnaire for patients with food allergy. They have been developed and validated, including an adult form (AF), child form (CF), and teenager form (TF) and 2 forms completed by the parents of food allergic children (Parent form PF and parent form teenagers PFT). These forms are reliable and valid instruments to measure the impact of food allergy on HRQOL (health related quality of life). More specifically, the Food Allergy Quality of Life Questionnaire (FAQLQ) assesses the effect of food allergy on the particpant's HRQOL (i.e., domains consist of risk of accidental exposure, emotional impact, allergen avoidance and dietary restrictions). The FAQLQ-Child Form (aged 8-12) (Flokstra-de Blok BM et al (2009) Clinical. Exp. Allergy; 39: (1) 127-37), FAQLQ-Teenager Form (aged 13-17) (Flokstra-de Block BM et al. (2008) J. Allergy Clinical Immunology; 122:139-44, 144.e1-2) and FAQLQ-Adult Form (>18 years of age) (Flokstra-de Blok BM et al (2009) Allergy; 64 (8): 1209-17), are self-administered, validated, food allergy-specific HRQOL questionnaires. The FAQLQ-parental form (FAQLQ-PF) is completed by parents of children aged 0-12 with food allergy (Dunn Galvin et al (2008), Clin Exp Allergy, 40 (2010), 476-485).

The number of items and domains varies by FAQLQ instrument administered. Each question is scored on a seven-point scale from 1 to 7 (i.e., from ‘no’ to ‘maximal’ impairment in HRQOL, respectively). The total score is the arithmetic average of all non-missing items. Domain scores are calculated similarly.

In yet another embodiment, the invention relates to LOU064 for use in the methods of the invention wherein the patient achieves a reduction from baseline in the total domain score FAIM.

FAIM (food allergy independent measure) is a validated and reliable instrument for measuring the patients' expectation of outcome and was developped for children (FAIM-CF), adolescent (FAIM TF) and adults (FAIM AF). (Flokstra-de Block et al. Allergy 65 (5): 630-5). The Food Allergy Independent Measure (FAIM) reflects the participant's perceived food allergy severity and food allergy-related risk. The FAIM includes 4 questions, also scored from 0 to 6, with higher scores indicating a worse expectation of adverse outcome if an allergen is accidentally ingested (e.g., a severe reaction or death).

In yet another embodiment, the invention relates to LOU064 for use in the methods described herein, wherein in a double-blind placebo-controlled food challenge with 600 mg of an allergen (e.g. peanut allergen), the observed difference in the responder rate between the treated and non-treated patients is superior to 35%, wherein the responder rate is defined as no more than a mild response to 600 mg oral food challenge.

In a further embodiment, the invention relates to LOU064 for use in the methods described herein, wherein the patient has no or no more than a mild reaction to oral food challenge (e.g. after receiving a dose of 600 mg of allergen (e.g. peanut protein). A mild response is defined as per consortium of food allergy research (CoFAR: Sampson HA et al. J. Allergy Clin. Immunol; 130 (6): 1260-74) which include one or more of the following:
In Skin-limited (few) or localized hives, swelling (e.g., mild lip edema), skin flushing (e.g., few areas of faint erythema) or mild pruritus (e.g., occasional scratching)
Respiratory-rhinorrhea (e.g., occasional sniffling or sneezing), nasal congestion, occasional cough, throat discomfort
GI-mild abdominal discomfort (including mild nausea with or without decreased activity), isolated emesis thought to be secondary to gag

In another embodiment, the invention relates to LOU064 for use in the methods described herein, wherein the patient does not experience a moderate nor a severe reaction to oral food challenge (e.g. after receiving a dose of 600 mg of allergen (e.g. peanut protein). A moderate response is defined as per consortium of food allergy research (CoFAR: Sampson HA et al. J. Allergy Clin. Immunol; 130 (6): 1260-74) which include one or more of the following:
Skin-systemic hives (e.g., numerous or widespread hives), swelling (e.g., significant lip or face edema), pruritus causing protracted scratching, more than a few areas of erythema or pronounced erythema
Respiratory-throat tightness without hoarseness, persistent cough, wheezing without dyspnea
GI-persistent moderate abdominal pain/cramping/nausea with decreased activity, vomiting

A severe response is defined as per consortium of food allergy research (CoFAR: Sampson HA et al. J. Allergy Clin. Immunol; 130 (6): 1260-74) which include one or more of the following:
Skin—severe generalized urticaria/angioedema/erythema
Respiratory—laryngeal edema, throat tightness with hoarseness, wheezing with dyspnea, stridor
GI—severe abdominal pain/cramping/repetitive vomiting
Neurological—change in mental status
Circulatory—clinically significant hypotension

### Safety

Short-term safety of LOU064 at single doses up to 600 mg and further at 100 mg b.i.d. for up to 18 days has been shown in Phase I clinical studies. However, no data concerning long-term safety are available at this time.

Considering dose-limiting side effects observed with the covalent irreversible BTK inhibitors evobrutinib and tolebrutinib, with evobrutinib showing dose-limiting liver enzyme elevations already at a dose of 75 mg b.i.d. in Phase II clinical studies and tolebrutinib showing dose-limiting diarrhea (Becker A. et al., 2019, Clin Transl Sci; 13,325-336; Montalban X. et al., 2019, N Engl J Med; 380 (25): 2406-17, Smith P.F. et al., 2019, ACTRIMS Forum, Feb. 28, 2019, P072), it is encouraging that no meaningful increase of the incidence of these adverse events was seen with LOU064 even at the higher dose. We have demonstrated that even at the higher dose of 100 mg b.i.d. over an extended period of time (up to 52 weeks), LOU064 is safe (Example 14). In particular, LOU064 does not induce any dose-limiting liver enzyme elevations and other off-target-effects at a dose of 100 mg b.i.d. over an extended period of time (up to 52 weeks). LOU064 is therefore suited for long-term treatment.

Therefore, one object of the invention is LOU064 for use in the described methods, wherein by week 12, by week 24 or by week 52 of treatment the levels of alanine aminotransferase (ALT), aspartate aminotransferase (AST), and lipase do not change by more than 10% as compared to the baseline level at the start of therapy.

It was therefore particularly surprising that LOU064 not only efficiently prevents IgE driven allergic reactions (e.g. food allergic reactions) but also possesses a better safety profile as compared to other BTK inhibitors, particularly as compared to acalabrutinib, especially when treatement is maintained over an extended period of time. For example, BTK inhibitors (ibrutinib, acalabrutinib, and zanubrutinib) which are currently approved primarily for the treatment of hematologic malignancies have known safety liabilities. Main safety liabilities include infections, effect on platelet function (risk for bleeding), and cytopenias. The other safety concerns for one or more approved BTKis include cardiac arrhythmia (atrial fibrillation and flutter) and, for ibrutinib only, cardiac failure and hypertension.

Hence, in a preferred embodiment of the present invention, LOU064 or a pharmaceutically acceptable salt thereof for use in treating or preventing IgE driven allergic reactions (e.g. food allergic reaction) is used in a long-term treatment. The term long-term treatment indicates that LOU064 or a pharmaceutically acceptable salt thereof is used over an extended period of time. For example, LOU064 or a pharmaceutically acceptable salt thereof can be used safely for more than 6 months, 10 months, 1 years, 2 years, 3 years, 4, years, 10 years. LOU064 or a pharmaceutically acceptable salt thereof might be used up to 2 years, 5 years, 10 years, 15 years, 20 years or for life.

In one embodiment, LOU064 not only efficiently prevents IgE driven allergic reaction (e.g. food allergic reaction) but has a safety profile including one or more of the following characteristics:
no clinically relevant increase of risk of infection,
no clinically relevant increase of major bleeding,
no clinically relevant elevation of liver enzymes; allowing for such long treatment.

It is anticipated that LOU064 oral drug exposure could be increased several fold when administered with CYP3A inhibitors, especially strong CYP3A inhibitors, e.g. strong CYP3A4 inhibitors. Likewise, strong inducers of CYP4A, e.g. of CYP3A4, may significantly decrease the exposure and lead to reduced efficacy of LOU064. These properties of LOU064 are not only relevant for the treatment and/or prevention of IgE driven allergic reaction but also for any BTK-mediated condition. Strong CYP3A inhibitors or CYP3A inducers are defined according to the FDA 2020 guidelines. Therefore, a strong CYP3A inhibitors (e.g. CYP3A4 inhibitors) are inhibitors which upon co-administration with LOU064 leads to an increase of the area under the curve (AUC) of more than 5 fold or a decrease of more than 80% in clearance as compared to administration of LOU064 alone. Strong CYP3A inducers (e.g. strong CYP3A4 inducers) are inducers which upon co-administration with LOU064 decrease the AUC by 80% or more (e.g. by 85%, by 90%, by 95%) as compared to administration of LOU064 alone.

Concomitant administration with strong CYP3A inhibitors and/or inducers, e.g. strong CYP3A4 inhibitors and/or inducers may possibly cause substantial changes in LOU064 drug exposure, and should be avoided. Strong CYP3A4 inhibitors include drugs selected from boceprevir, clarithromycin, cobicistat, conivaptan, danoprevir/ritonavir, darunavir/ritonavir, elvitegravir/ritonavir, idelalisib, indinavir, indinavir/ritonavir, itraconazole, ketoconazole, LCL161, lopinavir/ritonavir, mibefradil, nefazodone, nelfinavir, posaconazole, ritonavir, saquinavir, saquinavir/ritonavir, telaprevir, telithromycin, tipranavir/ritonavir, troleandomycin, Viekira pack or/and voriconazole. CYP3A4 inhibitors can also be contained in grapefruit juice.

Therefore, in another preferred embodiment LOU064 is not administered concomitantly with a strong inhibitor and/or inducer of CYP3A4e.g. as defined hereinabove.

It has further been found that LOU064 can be co-administered with oral contraceptives such as ethinylestradiol or levonorgestrel without a major impact on their exposure and efficacy. Therefore, in a preferred embodiment, LOU064 is co-administered with oral contraceptives.

In a preferred embodiment, no premedication is administered prior to the first dose of LOU064.

As a covalent irreversible BTK inhibitor LOU064 acts via irreversible inhibition of BTK which is countered by de novo protein synthesis. Thus, without wishing to be bound by any theory, it is believed that, whereas reconstitution of the B cell pool after B cell depletion can take several months, the restoration of B cell function after BTK inhibition can be achieved shortly after discontinuation, in particular within days. Therefore, if need be, this therapy could rapidly be stopped which provides clinicians and patients with easier and faster reaction capacity when unforeseen circumstances arise.

In another embodiment LOU064 is advantageously selected if the patient will undergo chemotherapy within the next 12 months.

Especially in light of the COVID-19 pandemic, B cell-depleted patients have a higher risk of infection. Furthermore, the absence of a fully functional adaptive immune response likely leads to a more severe course.

However, since LOU064 does not result in depletion of the pool of B cells, cessation of the therapy leads to a quick restoration of complete B cell function. This gives patients and treating physicians the possibility to quickly respond to infectious disease or vaccination requirements, in particular vaccination with live vaccines and attenuated vaccines.

According to the invention, LOU064 can be administered during an infection, e.g. during a COVID-19 infection. Thus, LOU064 administration can be continued during an infection, e.g. during a COVID-19 infection.

Preferably, LOU064 administration is delayed in patients with an active infection, e.g. COVID-19, until the infection is resolved.

Thus, one embodiment of the invention relates to LOU064 for use in the treatment or prevention of IgE driven allergic reaction (e.g. food allergic reaction), wherein a patient acutely or previously infected by COVID-19 is treated.

In a further embodiment LOU064 treatment is continued during COVID-19 infection.

In a preferred embodiment LOU064 treatment is interrupted during COVID-19 infection and continued after overcoming the infection.

A still further embodiment of the invention relates to LOU064 for use in the treatment or prevention of IgE driven allergic reaction (e.g. food allergic reaction), wherein the patient is vaccinated during LOU064 therapy. Alternatively, the patient can be vaccinated during LOU064 therapy with non-live vaccines. In one embodiment, the patient is vaccinated with quadrivalent Influenza vaccine, the PPV-23 vaccine or the KLH neoantigen vaccine during LOU064 therapy (e.g. at day 15 after initiating LOU064 therapy). In one aspect of this embodiment, the patient receiving quadrivalent Influenza vaccine, achieves a response as defined by a >4-fold increase of anti-hemmaglutinin antibody titers at 28 days after vaccination compared to baseline. In another aspect of this embodiment, the patient receiving the PPV-23 vaccine achieves a >2-fold increase of IgG titers 28 days after vaccination compared to baseline. In yet another embodiment, the patient receiving the KLH neoantigen vaccine achieves a T-cell dependent antibody response as measured by anti-KLH IgG and IgM titers 28 days after vaccination.

Another embodiment of the invention relates to LOU064 for use in the treatment or prevention of IgE driven allergic reaction (e.g. food allergic reaction), wherein LOU064 treatment is discontinued for vaccination, in particular wherein LOU064 treatment is discontinued 5-10 days, e.g. 7 or 8 days, preferably 6 weeks prior to vaccination and continued after vaccination, e.g. 5-20 days, preferably 5-10 days or most preferably 10-15 days after vaccination. In an alternative embodiment the vaccination is a vaccination with live vaccines and/or attenuated vaccines. In a particular aspect of this embodiment, the patient is vaccinated with quadrivalent Influenza vaccine, the PPV-23 vaccine or the KLH neoantigen vaccine after discontinuing LOU064 treatment (e.g. 5-10 days or 7 or 8 days after discontinuing LOU064 treatment). In one aspect of this embodiment, the patient receiving quadrivalent Influenza vaccine, achieves a response as defined by a >4-fold increase of anti-hemmaglutinin antibody titers at 28 days after vaccination compared to baseline. In another aspect of this embodiment, the patient receiving the PPV-23 vaccine achieves a >2-fold increase of IgG titers 28 days after vaccination compared to baseline. In yet another embodiment, the patient receiving the KLH neoantigen vaccine achieves a T-cell dependent antibody response as measured by anti-KLH IgG and IgM titers 28 days after vaccination. LOU064 treatment is then continued starting on Day 29 after vaccination.

### General

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise (e.g. reaction may include several reactions). Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure.

These examples should in no way be construed as limiting the scope of the disclosed subject matter, as defined by the appended claims.

### EXAMPLES

### Example 1: Passive Cutaneous Anaphylaxis (PCA) in Mice

LOU064 was tested in a murine PCA model. The model used BALB/c mice aged 8-10 weeks of age. For passive sensitization with hapten-specific IgE, mice under isoflurane anesthesia were given an intradermal injection of 20 μl anti-DNP IgE in saline into the right ear and 20 μl of saline into the left ear. For FccR-specific PCA, mice were given a low dose of 3 ng anti-DNP IgE. For maximal PCA mice were given a high dose of 25 ng anti-DNP IgE. LOU064 was administered p.o. in a volume of 5 ml/kg body weight. A first dose was administered 4 hours after sensitization on the evening before challenge. The next morning, 2 hours prior to hapten challenge, mice were given a second oral dose of LOU064.

Mice were challenged with hapten 2 hours after the second dose by placing them into a heated chamber. Animals were then injected i.v. with 100 μg of the hapten DNP-HSA (dinitrophenyl-human serum albumin) in 200 μl of 2% Evans blue diluted in PBS. The mice were then returned to their cage for exactly 30 minutes. Thirty minutes later, animals were then anesthetized with isoflurane and bled retro-orbitally using EDTA coated blood collection tubes for analysis of compound exposure. The mice were then sacrificed by cervical dislocation. A 6 mm punch biopsy was taken from both the left and the right ear and placed into 100 μl of formalin. The skin biopsies were incubated at 80° C. overnight to extract the dye. Twenty-four hours later 80 μl of formalin solution were transferred to a clear flat bottom 96 well plate and the optical absorption was read at 620 nm. Analysis was performed by subtracting the left ear (saline) from the right ear (IgE) to determine how much dye was released into the skin tissue of the biopsy. Statistical significance was determined by ANOVA. Spleens were removed for BTK occupancy analysis.

Analysis of blood level: Blood samples were taken as denoted and compound measurement was performed. Blood samples were spiked with a structurally similar internal standard mixed with acetonitrile (4 volumes) and centrifuged at ˜ 4000 rpm (4° C.). Supernatant was then transferred into a microtiter plate. An aliquot of each sample was injected into an LC-MS/MS system for determination of concentrations of the parent molecule.

The concentrations of LOU064 present in mouse blood, 2.5 hours after the second compound dose are shown for the low-dose IgE sensitization in Table 1.

TABLE 1
Concentrations of LOU064 in blood

| Dose (mg/Kg) | Concentration (nM) Mean ± SD (n = 5) |
|---|---|
| 3 | 7 ± 2 |
| 10 | 35 ± 14 |
| 30 | 111 ± 56 |
| 100 | 488 ± 371 |

Values represent the Mean ± SD blood concentrations from n = 5 animals

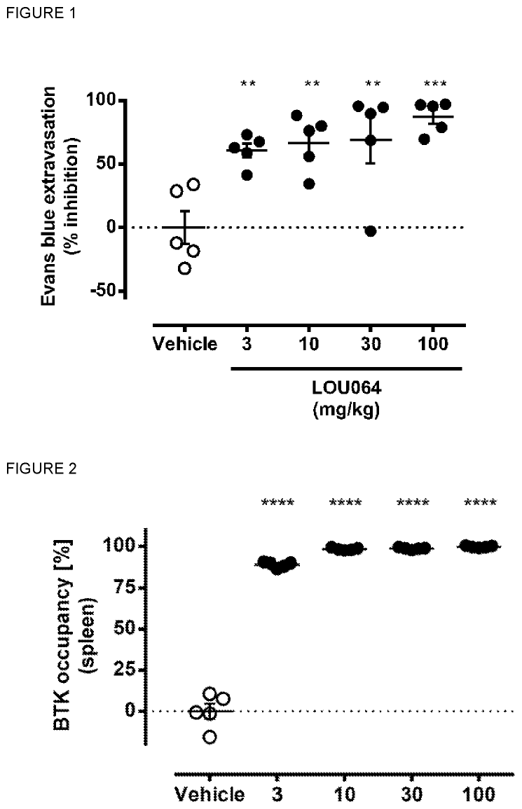

### Analysis of BTK Occupancy in Spleen (FIG. 2)

The levels of BTK occupancy after dosing of LOU064 was determined in separate ELISAs for total BTK protein and for bound BTK (=free BTK).

Covalent binding of compounds to BTK was determined with immunoassays for free BTK (i.e., not covalently occupied by compound) and total BTK protein. For free BTK measurements, samples were incubated with a biotinylated covalent BTK probe (Pulz R et al, ACS Med. Chem. Lett. 2019, 10, 1467-1472) then samples were added to streptavidin ELISA plates to allow binding of probe-bound BTK. The binding of the probe to BTK is mutually exclusive with compound binding to BTK. Plate-bound BTK was detected with an anti-BTK antibody (D3H5, Cell Signaling Technology). For total BTK measurements, an ELISA plate was coated with D3H5 anti-BTK to capture total BTK. An anti-BTK antibody (no. 53, BD Biosciences) directed to a different epitope was then used to detect captured BTK. The respective free BTK levels for each sample were normalized to the total BTK level in the same sample, and these ratios were expressed as percentage of the vehicle control samples.

For spleen BTK occupancy, each spleen was crushed and lysed in a GentleMACS (Miltenyi, #130-093-236) tube in 0.4 ml cold Pierce IP lysis buffer (Thermo Scientific #87787) containing a protease inhibitor cocktail (Complete, Roche #11 836 170 001) and 5 mM EDTA on ice during 10 minutes. Spleen lysates were cleared by centrifugation and aliquots were incubated with 10 UM of the covalent biotinylated BTK probe at room temperature for 2 hours. Two separate ELISA assays were run to determine the relative levels of total BTK protein and of the BTK-bound covalent probe (equivalent to free unoccupied BTK).

The relative BTK occupancy was calculated as the ratio of free BTK (signal of bound BTK) to total BTK for each sample: Ri=free BTK/total BTK. The individual Ri values were then normalized to the vehicle group and expressed as percent occupancy: (average of Ri vehicle group-Ri)/(average of Ri vehicle group)*100%

FIG. 2 shows the relative BTK occupancy in spleen 2.5 hours after the second oral dosing of LOU064. High and almost maximal levels of BTK occupancy were found for all dose-levels. The occupancy levels relative to vehicle controls were 89.1%+1.7 for 3 mg/kg, and 98.4 to 99.7% for the doses of 10 to 100 mg/kg.

### Conclusion:

Based on the above results, the BTK inhibitor, LOU064 is effective at inhibiting FccR responses on mast cells in the skin (FIG. 1). This study showed near complete inhibition of the anaphylactic response was observed at doses as low as 3 mg/kg. Blood concentrations of LOU064 2.5 hours after the second oral administration showed a dose proportional increase.

The blood exposures in both experiments were comparable. Spleen BTK occupancy measured at the same time point showed an almost complete target occupancy with a proportionality for the lowest dose. In conclusion, LOU064 has been demonstrated to inhibit skin anaphylaxis in the mouse PCA model in a significant manner after a two oral administrations

### Example 2: Reverse Passive Arthus (RPA) Reaction in Mouse Skin

The effect of a single dose of LOU064 was tested in the acute mouse reverse passive Arthus (RPA) model to assess its effect on a dermal anaphylaxis reaction triggered by IgG immune complexes and mediated by FcGRIIl on mast cell.

Female C57BI6 mice (Charles River, France), 8-10 weeks of age, were used. The back of the mice was shaved 24 hours prior to the skin injections. This allowed any potential irritation of the skin to resolve. The Arthus reaction was triggered two hours after compound dosing in the dose-response study or at the timepoints noted in the time-course study. To elicit the Arthus reaction, 50 μl of PBS (control site) or polyclonal rabbit anti-ovalbumin IgG at a concentration of 30 μg in PBS (C6534, Sigma) was injected i.d. into the dorsal skin. The intradermal injection was followed immediately by an i.v. injection of 200 μl ovalbumin (20 mg/kg) in saline (05450 Fluka). Three hours after the injection of the ovalbumin the mice were killed, bled for monitoring of the compound concentration and the spleens removed for BTK occupancy measurement. The thickness of the injected control and anti-ovalbumin IgG skin sites was measured using a digital caliper, and the thickness of the anti-ovalbumin IgG injected sites minus the saline control sites were then calculated for the mice in all treatment groups.

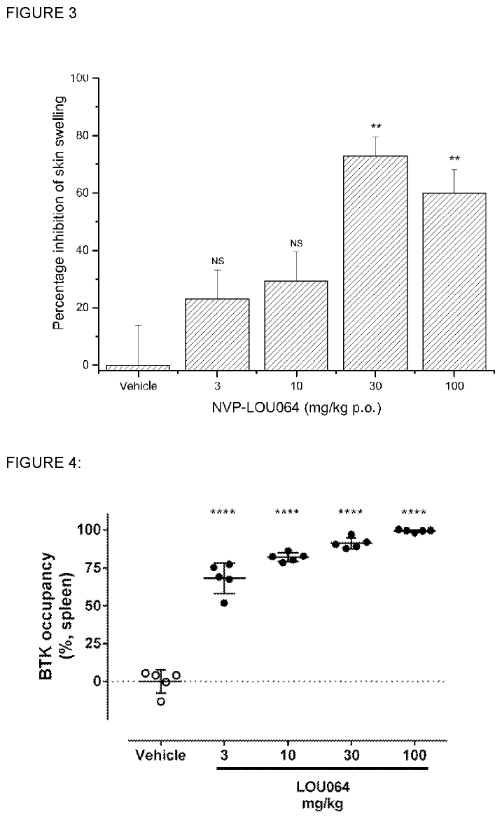

The effects of LOU064 treatment on skin swelling in mice, dosed as a single p.o. nanosuspension gavage at 3, 10, 30 and 100 mg/kg are shown in FIG. 3. In comparison to vehicle treated mice, LOU064, dosed 2 hours prior to the time of Arthus reaction induction, demonstrated a dose-related reduction in the skin swelling response at 3 hours. The inhibition of the Arthus reaction was statistically significant at the two higher doses of 30 and 100 mg/kg at 73.0%+14.6 and 61.2%+14.5, respectively.

### Analysis of LOU064 Levels in Blood

Blood samples were taken as denoted and compound measurement was performed. Blood samples were spiked with a structurally similar internal standard mixed with acetonitrile (4 volumes) and centrifuged at ˜ 4000 rpm (4° C.). Supernatant was then transferred into a microtiter plate. An aliquot of each sample was injected into an LC-MS/MS system for determination of concentrations of the parent molecule.

The concentrations of LOU064 present in mouse blood, 5 hours LOU064 dosing are shown in table 2:

| Dose (mg/Kg) | Concentration (nM) Mean ± SD (n = 5) |
|---|---|
| 3 | 2.04 (n = 1) |
| 10 | 2.87 ± 0.58 |
| 30 | 25.20 ± 20.89 |
| 100 | 139.57 ± 63.88 |

### BTK Occupancy Analysis:

For BTK occupancy in spleen, the experiment was performed as described in the PCA example. For lung BTK occupancy, lysates were prepared from complete lungs including tracheas after perfusing the organs with saline to remove blood in the same manner as for spleen (except that the lysis buffer volume was 500 μl). The lung lysates were then analyzed for free BTK in a separate ELISA. This consisted of a capture step with anti-BTK (Cell Signaling Technologies, #8547, carrier free). Then the captured BTK protein was incubated with 1 μM of covalent biotinylated BTK probe for 1 hour at room temperature. The BTK-bound biotinylated probe was detected by with a streptavidin-HRP conjugate (Cell Signaling Technologies, #3999, used at a dilution of 1:1′000) and a chemiluminescence substrate (Supersignal ELISA Pico, Thermo Scientific, #37070). The luminescence values were corrected for ELISA background reads from control samples measured in absence of BTK capture antibody for the total BTK ELISA and in absence of probe for the BTK-bound ELISA, respectively. The relative BTK occupancy was calculated as the ratio of free BTK (signal of the covalent biotinylated probe bound BTK) to total BTK for each sample: R=free BTK/total BTK. The individual Ri values were then normalized to the vehicle group and expressed as percent occupancy: (average of Ri vehicle group−Ri)/(average of Ri vehicle group)*100%.

FIG. 4 shows the relative BTK occupancy in spleen 5 hours after oral dosing of LOU064. A dose-dependent increase in BTK occupancy was seen, reaching a mean BTK occupancy of 68.1%±10.0 for 3 mg/kg, 82.1%±2.92 for 10 mg/kg, 91.3%±3.62 for 30 mg/kg and 99.3%±0.7 for 100 mg/kg.

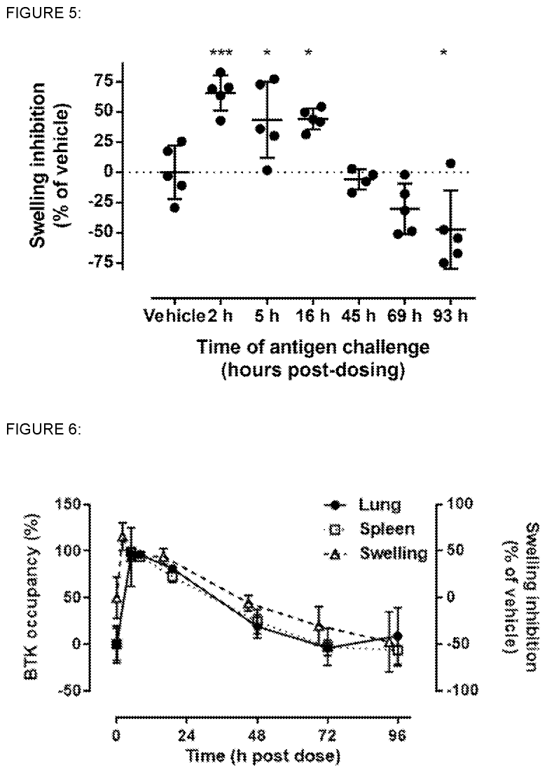

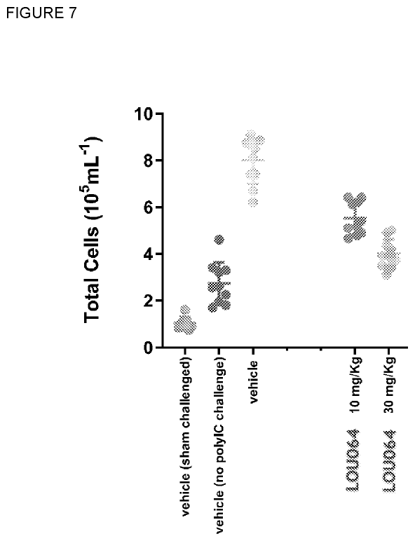

In a subsequent study we assessed the duration of the pharmacologic effect of LOU064 in the skin. When the Arthus reaction was elicited at the same timepoint as in the previous study at 2 hours post oral dosing of 30 mg/kg LOU064 the inhibition of skin swelling was maximal with 65.7% (+14.6). At this timepoint the effect was in a similar range as in the previous study. When the Arthus reaction was elicited at later timepoints it became apparent that the pharmacologic effect of LOU064 in the skin returned to the levels of the vehicle group by the 45 hours timepoint (FIG. 5).

From the same animals, the BTK occupancy in spleen and lung was analyzed at the termination timepoint (3 hours after eliciting the Arthus reaction). The BTK occupancy in spleen and lung followed a very similar kinetic compared to the pharmacological effect on skin swelling (FIG. 6).

Compound exposure was assessed for the first timepoints after the single dose of 30 mg/kg LOU064 (Table 3)

TABLE 3
blood exposure levels of LOU064 after
single oral dose of 30 mg/kg

| Timepoint (hours post-dose) | Concentration (nM) Mean ± SD (n = 5) |
|---|---|
| 5 | 28.4 ± 18.2 |
| 8 | 9.2 ± 6.7 |
| 19 | Below LLOQ |

### Conclusion:

This experiment showed the effects of LOU064 in the mouse RPA, a model for a delayed-type cutaneous anaphylaxis mediated by IgG immune complexes and proinflammatory FcγRs. Oral treatment with LOU064 at a single dose of 3, 10, 30 and 100 mg/kg, 2 hours prior to induction of the RPA response, reduced skin swelling in a dose-related manner 3 hours after immune complex challenge. The skin swelling inhibition was statistically significant at both the 30 and 100 mg/kg doses. Blood concentrations of LOU064 5 hours after oral dosing increased with dose, and showed some overproportionality. Whether this limited overproportionality reflects changes in Tmax or AUC is unknown. Spleen BTK occupancy measured 5 hours after dosing showed a dose-related increase and reached maximal levels. At the lower two doses of 3 and 10 mg/kg, spleen occupancy appeared to exceed the effects on skin swelling. This might reflect a slightly slower target occupancy kinetics after single dose of the less perfused skin compared to the highly perfused spleen. The pharmacologic effect of LOU064 after a single dose of 30 mg/kg was maximal when the Arthus reaction was elicited 2 hours after oral dosing. When the Arthus reaction was elicited at later timepoints the effect diminished and reached similar levels to the vehicle group at 45 hours post dose. The baseline drift of skin swelling inhibition to the last timepoint can be attributed to an increased response to the Arthus challenge across the groups over time. The vehicle group was measured on the first day at the same time as the 2 and 5 hour compound groups. Over the same time-course, the BTK occupancy was monitored in spleen and lung. In both tissues the BTK occupancy followed a very similar kinetic compared to the pharmacologic effect on skin swelling. In the absence of a method sufficiently sensitive to measure BTK occupancy in the skin, it might be inferred that skin BTK occupancy after a single oral dose of LOU064 follows a similar kinetic to the spleen occupancy and skin pharmacology. In conclusion, LOU064 has been demonstrated to inhibit skin swelling in the mouse reverse passive Arthus model in a significant and dose related manner after a single oral administration.

### Example 3: Ovalbumin Induced Anaphylaxis Mouse Model

This experiment was carried out to investigate the anti-inflammatory effect of LOU064 (10 mg and 30 mg/Kg in OVA-sensitized mice instilleed with polyinosine-polycytidylic acid (poly I: C).

### Study Plan:

### Group Size:

| | Sensitization | | | | i.t. 16 hrs |
|---|---|---|---|---|---|
| | (s.c.) | Aerosol | Pre-treatment (p.o., | | post final |
| | (days 0 & | challenge | 10 mL/Kg, starting day 21- | Dose | aerosol |
| Grp | 14) | (day 21) | 22) | Frequency | (day 24) |
| 1 | OVA | saline | Vehicle/Vehicle B.I.D | B.I.D/B.I.D | PBS |
| 2 | OVA | OVA | Vehicle/Vehicle B.I.D | B.I.D/B.I.D | PBS |
| 3 | OVA | OVA | Vehicle/Vehicle B.I.D | B.I.D/B.I.D | Poly I:C |
| 4 | OVA | OVA | LOU064 (10 mg/Kg)/Vehicle | B.I.D/B.I.D | Poly I:C |
| 5 | OVA | OVA | LOU064 (30 mg/Kg)/Vehicle | B.I.D/B.I.D | Poly I:C |

### Animals:

Male Balb/c mice (20-30 g, Charles Rivers UK Ltd) were housed for 7 days prior to commencement of the study in cages of 5 and were subject to a 12:12 hr light dark cycle. Mice were fed a standard mouse chow and water was available ad libitum.

### Allergen Exposure:

Mice were actively sensitised with ovalbumin (15 μg, s.c.) and 25 μL of Imject Alum on days 0, 7 and 14. In order to elicit a local inflammatory response in the lung, mice were repeatedly exposed on days 21-24 with an aerosol of 1% w/v ovalbumin in phosphate buffered saline (PBS), generated with an ultrasonic nebuliser (Aerogen) for 20 min. 16 h after the final exposure to OVA on day 24, polyinosine-polycytidylic acid (poly I: C, 100 μg/animal) or the vehicle (PBS) was delivered intratracheally with a Microsprayer (model IA-1C with FMJ-250 high pressure syringe, Penn-Century) under anaesthesia with ketamine (100 mg/Kg, i.p.) and xylazine (10 mg/Kg, i.p.).

### LOU064 and Vehicle Treatment:

Mice were dosed with LOU064 or vehicle (0.5% Tween80/0.5% Methylcellulose (400 cP)/99% water) p.o. B.I.D and then received a single dose of vehicle (67% PEG200/33% PBS) 1 hour prior to Poly I: C.

Dosing Schedule for LOU064 and vehicle

| Day | | | Day | | | Day | | | Day | | | | Day |
|---|---|---|---|---|---|---|---|---|---|---|---|---|---|
| 21 | | | 22 | | | 23 | | | 24 | | | | 25 |
| 18.00 | 19.00 | 6.00 | 18.00 | 19.00 | 6.00 | 18.00 | 19.00 | 6.00 | 18.00 | 19.00 | 6.00 | 11.00 | 19.00 |
| Dose | OVA | Dose | Dose | OVA | Dose | Dose | OVA | Dose | Dose | OVA | Dose | Poly | BAL F |
| 1 | | 2 | 3 | | 4 | 5 | | 6 | 7 | | 8 | IC or | |
| | | | | | | | | | | | | PBS | |

### Bronchoalveolar Lavage (BAL) and Cell Counts:

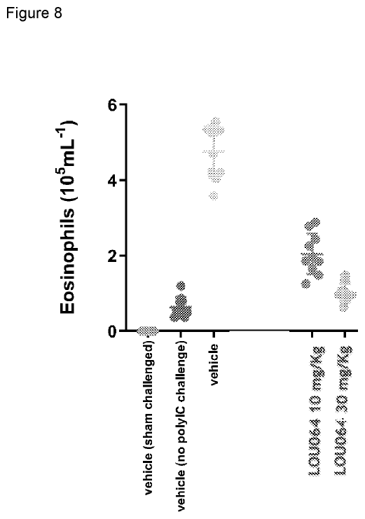

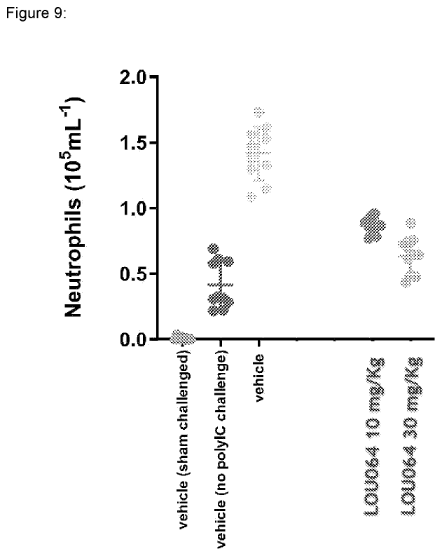

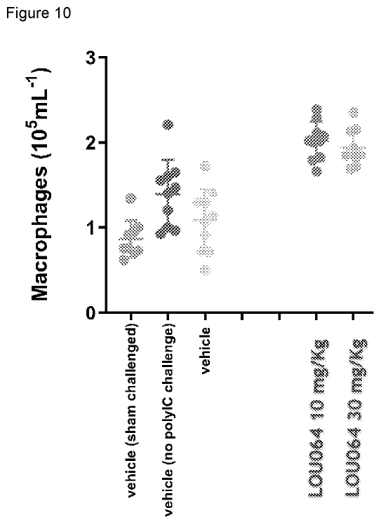

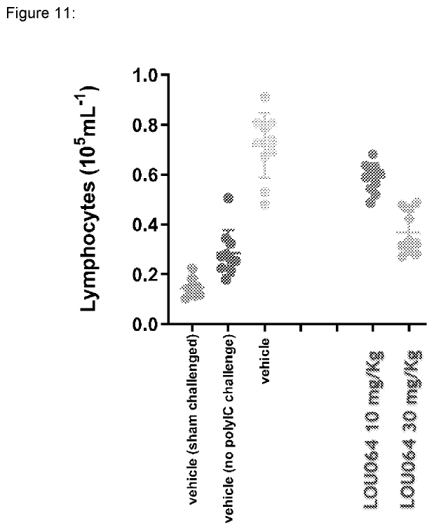

24 h after the final OVA challenge, blood samples were collected by venepuncture into pre-chilled Li Hep tubes. Each sample was mixed gently and kept on wet ice, for a maximum of 15 minutes, before centrifugation (1500 g, 10 min at 4° C.) to prepare the plasma. Each sample was aliquoted and stored at-800C until shipment. Immediately after blood collection, the animals were culled by an overdose with pentobarbital. The trachea was then isolated by a midline incision in the neck and separation of the muscle layers. A small incision was made into the trachea and a plastic cannula was inserted and secured in place with a suture. The airway was then lavaged by flushing out the lungs using 0.5 mL of phosphate buffered saline. This procedure was repeated until the recovered volume was 1.6 mL. The isolated BALF was then centrifuged at 1500 rpm for 10 mins at 4° C. and the supernatant was aliquoted (400 μL) at −80° C. for any future cytokine analysis. The cell pellets were then re-suspended in 1.6 mL of phosphate buffered saline and the BAL cells were then analysed for total and differential numbers. Total and differential cell counts of the BAL fluid samples were measured using a XT-2000iV analyser (Sysmex). Cell types differentially classified were eosinophils (FIG. 8), neutrophils (FIG. 9), or mononuclear cells (macrophages (FIG. 10) and lymphocytes (FIG. 11).

Conclusions: Treatment with LOU064 (10 mg/kg, 30 mg/Kg) significantly reduced numbers of cells involved in lung anaphylaxis (eosiphils, basophils, macrophages and lymphocytes)

### Example 4: Inhibition of FcER-Induced Mast Cell Degranulation in LAD2 Cells

Rationale and method: The human mastocytosis cell line LAD2 expresses FcERI and upon crosslinking of the receptor the cells degranulate and release inflammatory mediators like β-tryptase and hexosaminidase (Wernersson et al. 2014, Immunology; 14 (7): 478-94). The assay involves 4-hydroxy-3-iodo-5-nitrophenylacetic acid (NIP) haptenized bovine serum albumin (BSA) and the NIP-specific chimeric IgE antibody JW8 (Neuberger, M.S. et al. (1985), Nature 314 (6008), pp 268-70. doi: 10.1038/314268a0). The LAD2 cells were sensitized with JW8 IgE antibody and then incubated with serial compound dilutions for 30 minutes at 37° C. Then degranulation was triggered by adding haptenized NIP-BSA to crosslink the FcER. After one hour, supernatants were collected and analyzed for B-tryptase levels.

Result: In keeping with its potent activity on the basophil activation assay, LOU064 showed potent inhibition of IgE/FcER-induced mast cell degranulation in LAD2 cells with an IC50 of 7.0 nM+2.6 (n=3)

### Example 5: Inhibition of FcER-Induced Degranulation of Peripheral Blood Mononuclear

cells (PBMC)-derived mast cells Rationale and method: Primary human mast cells were derived from CD34-positive progenitor cells isolated from PBMC ((Neuberger, M.S. et al. (1985), Nature 314 (6008), pp 268-70. doi: 10.1038/314268a0). Blood from healthy volunteers was provided under informed consent and collected through the Novartis Tissue Donor Program in accordance with the Swiss Human Research Act and approval of the responsible ethic committee (Ethikkommission Nordwest-und Zentralschweiz). Briefly, CD34-positive cells were isolated from peripheral blood by positive selection and cultured in medium containing SCF, IL-6 and IL-3. After a 6 week differentiation culture, mast cells were harvested and characterized phenotypically for expression of c-Kit (CD117) and FcERI. The degranulation is based on NIP haptenized BSA and the NIP-specific chimeric IgE antibody JW8 (described in RD-2013-00412). The mast cells were sensitized with JW8 IgE antibody and then incubated with serial compound dilutions for 30 minutes at 37° C. Then degranulation was triggered by adding haptenized NIP-BSA to crosslink the FcER. After one hour, supernatants were collected and analyzed for β-tryptase levels.

Result: In similar manner as the other FcER inhibitory activity, LOU064 potently and completely blocked in vitro degranulation of primary human PBMC-derived mast cells with an IC50 of 5.7 nM+2.4 (n=7 donors).

### Example 6: Inhibition of Skin-Derived Human Mast Cell

Rationale and method: Skin tissue was used as a source of primary human tissue mast cells. Surgical skin discard samples were provided under informed consent and collected by the University Hospital Basel in accordance with the Swiss Human Research Act and approval of the responsible ethic committee (Ethikkommission Nordwest und Zentralschweiz). Skin tissue was dissociated and intact mast cells were prepared according to the protocol of Kulka and Metcalfe (Kulka et al. 2010, current protocols in Immunology; SUPPL. 90:1-11). The isolated mast cells were characterized phenotypically and used for a degranulation assay as described above ((Neuberger, M.S. et al. (1985), Nature 314 (6008), pp 268-70. doi: 10.1038/314268a0). The mast cells were sensitized with JW8 IgE antibody and then pre-incubated with serial compound dilutions for 30 minutes at 37° C. Then degranulation was triggered by adding haptenized NIP-BSA to crosslink the FcERI. After 30 minutes, supernatants were collected and analyzed for histamine with an HTRF-based immunoassay (Cisbio Bioassays).

Result: Similarly to the activity on the previous types of FcERI-induced cell signaling, LOU064 potently inhibited histamine-release from human primary skin-derived mast cells with an IC50 of 16.5 nM+5.0 (n=2 donors)

The in vitro cellular potency of LOU064 which was assessed across several cell types in examples 4-6 supports its use in allergic diseases driven by the FcER pathway.

### Example 7: Dose of LOU064 (Rationale)

It was demonstrated in a mice PCA model that LOU064 is effective at inhibiting FccR responses on mast cells in the skin (FIG. 1) at doses as low as 3 mg/kg. Spleen BTK occupancy which may be more relevant biomarkers for predicting efficacy in the prevention and/or treatment of IgE driven allergic reaction (particularly food allergic reaction) shows a nearly complete occupancy at 10 and 100 mg/kg.

It was further demonstrated that in mice, BTK occupancy in spleen and lung followed a very similar kinetics (FIG. 6).

It was also demonstrated that LOU064 is effective on reducing skin swelling in mice RCA model, mostly at higher dose (with an inhibition of the Arthus reaction of 73% at 30 mg/Kg).

Importantly, it was also demonstrated that LOU064 treatment of mice in an ovabulmin induced anaphylaxis model resulted in a significant reduction of the number of cells involved in lung anaphylaxis at a dose of 10 mg/kg and 30 mg/kg.

Overall pre-clinical results support an efficacious dose for LOU064 in mice as low as 10 mg/kg. 30 mg/kg has also shown to be efficacious in mice.

Additional BTK occupancy in human in spleen has been predicted using a translational PK/PD model in order to further assist dose selection.

### Prediction of BTK Occupancy Using a Translational PK/PD Model for LOU064

BTK occupancy in blood is not an informative biomarker for the purpose of dose selection due to LOU064 pharmacological properties (irreversible binding). It reaches full occupancy even at low doses before showing pharmacological activity through other biomarkers (CD63, CD203c, skin-prick test). Occupancy in tissue may be more representative of the expected efficacy of LOU064.

### Objectives

The purpose of this analysis was to characterize the pharmacokinetics (PK) of LOU064 in healthy volunteers and to use a previously developed, translational target occupancy model to simulate BTK occupancy in human spleen/tissues across a range of doses and dosing regimen (B.I.D vs QD).

### Data

Pharmacokinetic data from a Phase I clinical study reported by Kaul et al. (2021) were used in the current analysis, including 102 patients.

### Methods

A translational target occupancy model to simulate BTK occupancy in spleen/tissues was developed using a two-step approach.

In a first step, a population PK model was established to describe LOU064 PK data from Phase I clinical study reported by Kaul et al. (2021). In a second step, the parameter estimates from the population PK model were used in the BTK occupancy model to predict BTK occupancy in blood and spleen/tissues. Ultimately the BTK occupancy model was used to predict the BTK occupancy in spleen/tissues for different dosing regimens (QD, B.I.D) at different doses.

### Results

A population PK model has been developed to describe the interim PK from a Phase I clinical study reported by Kaul et al. (2021). In order to address the change in clearance after repeated dosing for doses lower than 50 mg (lower clearance at steady state at Day 12 when compared to Day 1 with no difference at higher doses), the clearance was modeled as a function of exponential time decay for doses less than 50 mg and a constant clearance for doses above 50 mg. Overall the resulting population model described the PK data reasonably well.

The PK parameter estimates were used in a translational BTK occupancy model to simulate BTK occupancy at steady state. The BTK occupancy simulations showed that B.I.D dosing is more effective than QD dosing at the same dose to achieve higher BTK occupancy (at trough or averaged over 24-hour interval).

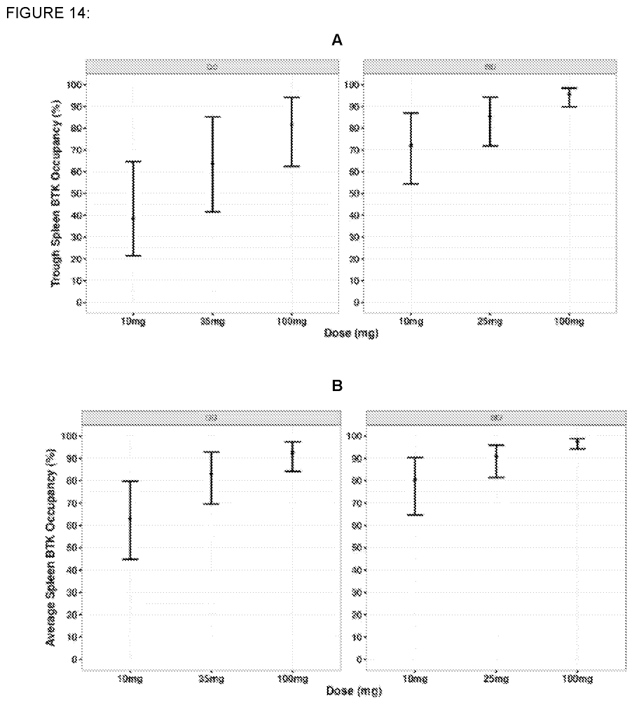

For a selected number of doses at QD and B.I.D regimen, steady-state BTK occupancy at trough and averaged over a period of 24 hours are shown in FIG. 14A (Trough over 24 hours of BTK Occupancy at steady state) and FIG. 14B (Average over 24 hours of BTK Occupancy at steady state), respectively for dosing regimens of 10 mg, 35 mg, 100 mg once daily and 10 mg, 25 mg and 100 mg twice daily. Both figures show that a daily dose up to 200 mg (100 mg B.I.D) may be required to achieve a trough BTK occupancy ≥80% in a peripheral target tissue.

Simulations were performed to compare different dosing regimens. A comparison of simulated spleen BTK occupancy at steady state of 100 mg B.I.D vs. 100 mg QD over time is shown in FIG. 13. The graph shows that the occupancy from B.I.D dosing is higher and less variable compared to QD dosing, as expected from basic principles.

### Conclusions:

The BTK occupancy simulations showed that B.I.D dosing is more effective than QD dosing at the same dose to achieve higher BTK occupancy (at trough or averaged over 24-hour interval).

Based on a conversion model between animals and human (Journal of basic and clinical pharmacy, 7 (2), 27-31), the calculated human equivalent dose (HED) for 30 mg/kg corresponds to ˜ 170 mg for a 70 kg person and the calculated human equivalent dose (HED) for the lowest effective dose of 10 mg/kg correspond to ˜ 56 mg for a 70 Kg person (Nair, A. B., & Jacob, S. (2016).

Finally, according to a prediction model of spleen BTK occupancy in human, a b.i.d. dosing was shown to be more effective than QD dosing at the same dose to achieve higher BTK occupancy (FIG. 13). Accordingly, a dose of LOU064 of 25 mg b.i.d is the minimal human effective dose and a dose of 100 mg b.i.d. is also shown to be effective in human.

### Example 8: Clinical Study Peanut Allergy Phase 2

A randomized, double-blind, placebo controlled, 4 week study assessing three dosing regimens (LOU064 10 mg B.I.D, 25 mg B.I.D and 100 mg B.I.D) in adult patients with demonstrated peanut allergy.

This “peanut study” is evaluating the efficacy of LOU064 (10 mg B.I.D, 25 mg B.I.D, and 100 mg B.I.D) compared to placebo, in the proportion of patients who do not exhibit an objective allergic reaction to the double-blind, placebo controlled, oral food challenge (DBPCFC) at 600 mg of peanut protein after 4 weeks of treatment. Additionally, patients treated with only one week of LOU064 25 mg B.I.D will be compared to placebo, again assessing the proportion of patients who do not exhibit an objective allergic reaction to the DBPCFC at 600 mg of peanut protein. Secondary endpoints for all treatment arms include response to 1000 mg and 3000 mg of peanut protein.

### Example 9: Clinical Study Peanut Allergy Phase 3

A randomized, double-blind, placebo controlled, 10-18 week study assessing LOU064 in approximately 300 adults and adolescents (12-55 yrs) with demonstrated peanut allergy. The peanut study will evaluate the efficacy of LOU064 compared to placebo in the proportion of participants who do not exhibit and objective allergic reaction to the double-blind, placebo controlled, oral food challenge (DBPCFC) at levels of 600 mg, 1000 mg and 3000 mg of peanut protein at week 1 or 2.

### Example 10: Clinical Study Milk and Egg Allergy

A randomized, double-blind, placebo controlled, 10-18 week basket study assessing LOU064 in approximately 450 adults and adolescents participants 12 to 55 year old with demonstrated tree nuts, milk or shrimp allergies.

The basket study assessing tree nuts, milk and shrimp allergens is evaluating the efficacy of LOU064 compared to placebo, in the proportion of patients who do not exhibit an objective allergic reaction to the DBPCFC at multiple levels of 1′000 mg (no cumulative) and 3′000 mg (no cumulative) of tree nuts/milk/shrimp proteins at week 1 or 2.

### Example 11: 100 mg Film Coated Tablet

| | Amount per 100 mg | |
|---|---|---|
| | Film-coated | |
| Ingredient | tablet (mg) | Function |
| Tablet core | | |
| LOU064 | 100.0 | Drug substance |
| Mannitol | 243.8 | Carrier |
| Cellulose, microcrystalline/ | 85.8 | Diluent |
| Microcrystalline Cellulose | | |
| Copovidone | 50.0 | Binder |
| Croscarmellose sodium | 31.2 | Disintegrant |
| Sodium Stearyl Fumarate | 5.2 | Lubricant |
| Sodium laurilsulfate/Sodium | 4.0 | Surfactant |
| lauryl sulfate | | |
| Water, purified/Purified | — | Suspending agent/ |
| water¹ | | Solvent |
| Core tablet weight | 520.0 | |
| Coating | | |
| Basic coating premix, yellow | 14.2 | Film-coating |
| Basic coating premix, red | 4.4 | Film-coating |
| Basic coating premix, white | 4.4 | Film-coating |
| Basic coating premix, black | 1.2 | Film-coating |
| Water, purified² | — | Granulation liquid |
| Total film-coated tablet | 544.2 | |
| weight | | |

^{1, 2}Removed during processing

### Example 12:25 mg Film Coated Tablet

| | Amount per 25 mg | |
|---|---|---|
| | Film-coated | |
| Ingredient | tablet (mg) | Function |
| Tablet core | | |
| LOU064 | 25.0 | Drug substance |
| Mannitol | 60.9 | Carrier |
| Cellulose, microcrystalline/ | 21.5 | Diluent |
| Microcrystalline Cellulose | | |
| Copovidone | 12.5 | Binder |
| Croscarmellose sodium | 7.8 | Disintegrant |
| Sodium Stearyl Fumarate | 1.3 | Lubricant |
| Sodium laurilsulfate/Sodium | 0.9 | Surfactant |
| lauryl sulfate | | |
| Water, purified/Purified | — | Suspending agent/ |
| water¹ | | Solvent |
| Core tablet weight | 130.0 | |
| Coating | | |
| Basic coating premix, yellow | 3.6 | Film-coating |
| Basic coating premix, red | 1.1 | Film-coating |
| Basic coating premix, white | 1.1 | Film-coating |
| Basic coating premix, black | 0.3 | Film-coating |
| Water, purified² | — | Granulation liquid |
| Total film-coated tablet | 136.0 | |
| weight | | |

^{1, 2}Removed during processing

### Example 13:10 mg Film Coated Tablet

| | Amount per 25 mg | |
|---|---|---|
| | Film-coated | |
| Ingredient | tablet (mg) | Function |
| Tablet core | | |
| LOU064 | 10.0 | Drug substance |
| Mannitol | 60.1 | Carrier |
| Cellulose, microcrystalline/ | 8.1 | Diluent |
| Microcrystalline Cellulose | | |
| Copovidone | 5.0 | Binder |
| Croscarmellose sodium | 5.4 | Disintegrant |
| Sodium Stearyl Fumarate | 0.9 | Lubricant |
| Sodium laurilsulfate/Sodium | 0.4 | Surfactant |
| lauryl sulfate | | |
| Water, purified/Purified | — | Suspending agent/ |
| water¹ | | Solvent |
| Core tablet weight | 90.0 | |
| Coating | | |
| Basic coating premix, yellow | 2.9 | Film-coating |
| Basic coating premix, red | 0.9 | Film-coating |
| Basic coating premix, white | 0.9 | Film-coating |
| Basic coating premix, black | 0.25 | Film-coating |
| Water, purified² | — | Granulation liquid |
| Total film-coated tablet | 95.0 | |
| weight | | |

^{1, 2}Removed during processing

### Example 14: Safety of LOU064

The safety of LOU064 has been tested in Phase I and Phase II pharmacokinetic and clinical pharmacology healthy subject studies and in Phase II/Phase III clinical studies conducted with patients suffering from indications other than MS, particularly chronic spontaneous urticaria (CSU) and Sjoegren's Syndrome (SjS).

### Short-Term Safety of LOU064 in Phase I Clinical Study

Short-term safety of LOU064 as a single dose or as multiple doses for up to 18 days covering the dose range from 0.5 mg to 600 mg for up to 18 days and further at 100 and 200 mg b.i.d. for up to 12 days has been shown in Phase | clinical studies (Kaul, M. et al. (2021). Remibrutinib (LOU064): A selective potent oral BTK inhibitor with promising clinical safety and pharmacodynamics in a randomized phase | trial. Clinical and Translational Science. 10.1111/cts. 13005).

### Summary of Safety in Phase 2b Study (Extension Phase) in CSU Subjects (Interim Results)

In a 52-week open label extension study to evaluate the long-term safety and tolerability of LOU064 in eligible subjects with CSU who participated in the Phase 2b study the dose used was 100 mg b.i.d.

No safety signal has been observed based on an interim analysis of the 100 subjects who received at least 1 dose of LOU064 with a median exposure of 17.86 weeks (range: 2.9 weeks to 44.7). At the time of cut-off, 93 subjects (93%) were ongoing, and 7 subjects had discontinued from the study; none of the discontinuations were due to adverse events. Table 4 presents the safety summary observed in the Phase 2b study up to the cut-off date for the interim analysis.

TABLE 4
Interim analysis for open label extension study: Deaths,
other serious or clinically significant adverse events
or related discontinuations (Safety Set)

| | LOU064 |
|---|---|
| | 100 mg B.I.D. |
| | N = 100 |
| | n (%) |
| Patients with AE(s) | 58 (58) |
| Patients with serious or other significant | |
| events | |
| Death | 0 |
| Non-fatal SAE | 3 (3.0) |
| Discontinued study due to any AE(s) | 0 |
| Discontinued study due to an SAE(s) | 0 |
| Treatment interruption due to AE(s) | 5 (5.0) |
| Treatment interruption due to SAE(s) | 1 (1.0) |

Interim analysis cut off 31 Aug. 2020

Fifty-eight subjects (58%) experienced at least one treatment emergent AE. The majority of AEs were non-serious, did not lead to treatment discontinuation and were mild in severity. The most frequently affected SOC were Infections and infestations (14%) followed by Skin and subcutaneous tissue disorders (13%) with no trends with respect to specific adverse events. The most common adverse event preferred terms (≥2%) were headache (6%), diarrhea (4%), dizziness (3%) and gastroenteritis (3%); no bleeding events (defined as events under Haemorrhages SMQ broad and the PTs including Platelet aggregation abnormal, Platelet aggregation decreased, Platelet aggregation inhibition, Platelet dysfunction, Platelet function test abnormal and Platelet toxicity) or events under SOC Blood and lymphatic system disorders were reported. Three SAEs were reported: ovarian cyst, chest pain and appendicitis; none were considered related to study drug.

Conclusions from the Phase 2b Study and Corresponding Open Label Extension Study

Taken together, there have been no safety findings in the Phase 2b study, across all the doses assessed. Furthermore, in the corresponding CSU extension study, which uses LOU064 100 mg b.i.d. open-label, no safety signals have been observed in 100 subjects enrolled as of 31 Aug. 2020. The proposed highest dose of 100 mg LOU064 b.i.d. is considered to be well tolerated and with a favorable safety profile.

Summary of Safety in Phase 2b Study (Extension Phase) in CSU Subjects (Interim Results/Patients with Medium Exposure of 35.14 Weeks)

In the above 52-week open label extension study to evaluate the long-term safety and tolerability of LOU064 in eligible subjects with CSU who participated in the Phase 2b study with a dose of 100 mg b.i.d., a new interim analysis was performed with patients (N=183) with a medium exposure of 35.14 weeks and the results were compared to the safety results in the randomized double-blind, placebo-controlled Ph2b core study, in adult patients with CSU who received (1:1:1:1:1:1:1) remibrutinib 10 mg qd (once daily), 35 mg qd, 100 mg qd, 10 mg bid (twice daily), 25 mg bid, or 100 mg bid or placebo up to 12 weeks (wks) (NCT03926611). (Table 5)

In the long-term exposure of the ES (median 35.14 wks, N=183), the proportion of patients with at least one adverse effect (AE) on remibrutinib treatment (57.4% [n=105]) was similar to the CS (presented through for any remibrutinib dose) (58.1% [n=155]; median 12.14 wks, N=267). In the ES, there were 4 serious adverse effects (SAEs), 6 AEs leading to treatment discontinuation and no deaths. The incidences of AEs by primary organ class (SOC) reported in the ES and CS were similar: infections and infestations (23.0% and 24.0%), followed by skin/subcutaneous tissue disorder (17.5% and 16.9%) (Table 5). Incidences of reported AEs by preferred term were comparable in ES and CS with headache (6.6% and 9.7%) being most common. Incidence of AESI in the ES such as infections (23%), bleeding (4.4%) and cytopenias (0.5%) were in line with the CS. Newly occurring notable transaminase increases were single in both ES (isolated ALT>3xULN, normalized within 4 weeks, in 1 patient discontinued early for personal reasons) and CS (ALT>5xULN in 1 patient, normalised on treatment). The analysis of laboratory parameters did not reveal significant safety concerns and no clinically meaningful changes in vital signs were observed. There were no significant ECG findings or QT of >500 ms noted in any patient.

### CONCLUSION

Remibrutinib showed a favorable safety profile across the whole dose range with no new safety signals observed over longer-term exposure to 100 mg bid dose up to 52 wks in patients with CSU.

TABLE 5
Safety profile of remibrutinib(LOU064) in Phase 2b core and extension study (safety set)

| Core study (CS) | | | | | | | | | ES |
|---|---|---|---|---|---|---|---|---|---|
| | LOU064 | | | | | | | | LOU064 |
| | 10 mg | 35 mg | 100 mg | 10 mg | 25 mg | 100 mg | Any | | 100 mg |
| Patients | q.d. | q.d. | q.d. | b.i.d. | b.i.d. | b.i.d. | dose | Placebo | b.i.d. |
| n (%) | N = 44 | N = 44 | N = 47 | N = 44 | N = 43 | N = 45 | N = 267 | N = 42 | (N = 183) |
| Patients | 29 | 23 | 27 | 21 | 26 | 29 | 155 | 18 | 105 |
| with ≥1 AE | (65.9) | (52.3) | (57.4) | (47.7) | (60.5) | (64.4) | (58.1) | (42.9) | (57.4) |
| Discontinued | 0 | 0 | 0 | 3 | 1 | 3 | 7 | 0 | 6 |
| study treatment | (0.0) | (0.0) | (0.0) | (6.8) | (2.3) | (6.7) | (2.6) | (0.0) | (3.3) |
| due to AE(s) | | | | | | | | | |
| Patients with | 1 | 0 | 0 | 2 | 2 | 0 | 5 | 0 | 4 |
| SAE(s) | (2.3) | (0.0) | (0.0) | (4.5) | (4.7) | (0.0) | (1.9) | (0.0) | (2.2) |
| Most frequent AEs by primary system organ class (≥10% of all patients receiving any | | | | | | | | | |
| LOU064 dose or placebo group in CS or in 100 mg b.i.d. in ES) | | | | | | | | | |
| Infections and | 12 | 9 | 14 | 6 | 12 | 11 | 64 | 9 | 42 |
| infestations | (27.3) | (20.5) | (29.8) | (13.6) | (27.9) | (24.4) | (24.0) | (21.4) | (23.0) |
| Skin and | 7 | 9 | 5 | 6 | 12 | 6 | 45 | 2 | 32 |
| subcutaneous | (15.9) | (20.5) | (10.6) | (13.6) | (27.9) | (13.3) | (16.9) | (4.8) | (17.5) |
| tissue disorders | | | | | | | | | |
| Nervous system | 3 | 10 | 7 | 4 | 6 | 5 | 35 | 7 | 19 |
| disorders | (6.8) | (22.7) | (14.9) | (9.1) | (14.0) | (11.1) | (13.1) | (16.7) | (10.4) |
| Gastrointestinal | 7 | 4 | 6 | 6 | 2 | 5 | 30 | 5 | 26 |
| diseases | (15.9) | (9.1) | (12.8) | (13.6) | (4.7) | (11.1) | (11.2) | (11.9) | (14.2) |
| Most frequent AEs by PT | | | | | | | | | |
| (≥5% of patients in dose or placebo group in CS or in 100 mg b.i.d. in ES) | | | | | | | | | |
| Headache | 1 | 7 | 4 | 3 | 6 | 5 | 26 | 6 | 12 |
| | (2.3) | (15.9) | (8.5) | (6.8) | (14.0) | (11.1) | (9.7) | (14.3) | (6.6) |
| Nasopharyngitis | 7 | 2 | 2 | 4 | 4 | 4 | 23 | 3 | 6 |
| | (15.9) | (4.5) | (4.3) | (9.1) | (9.3) | (8.9) | (8.6) | (7.1) | (3.3) |
| Chronic | 3 | 2 | 3 | 4 | 2 | 2 | 16 | 1 | 6 |
| spontaneous | (6.8%) | (4.5%) | (6.4%) | (9.1%) | (4.7%) | (4.4%) | (6.0%) | (2.4%) | (3.3) |
| urticaria | | | | | | | | | |

### Example 15: Evaluation of the Modulation of Immune Response to Three Different Types of Vaccines by Concomitant and Interrupted Administration of Remibrutinib in Health Subject

Objectives and related endpoints

| Objective(s) | Endpoint(s) |
|---|---|
| Primary objective(s) | Endpoint(s) for primary objective(s) |
| To evaluate non-inferiority of concomitant and | Achievement of response where |
| interrupted remibrutinib treatment on the | response is defined as: |
| immune response following vaccinations in | Influenza: >4-fold increase of |
| healthy participants relative to placebo for a: | anti- hemagglutinin antibody titers |
| T cell-dependent vaccine (Seasonal | at 28 days (Day 43) after |
| Influenza, quadrivalent vaccine) | vaccination compared with |
| T cell-independent vaccine (PPV-23, | baseline (i.e. sero-conversion) |
| Pneumovax ®, Merck & Co. Inc., USA) | PPV-23: >2-fold increase of |
| | immunoglobulin G (IgG) titers 28 |
| | days (Day 43) after vaccination |
| | compared with baseline. |
| Secondary objective(s) | Endpoint(s) for secondary objective(s) |
| To assess the effect of concomitant and | T-cell dependent antibody response |
| interrupted remibrutinib treatment on the | as measured by anti-KLH IgG and |
| immune response following vaccinations in | IgM titers 28 days after vaccination |
| healthy participants relative to placebo for T | (Day 43) |
| cell-dependent de novo vaccine (KLH, | All safety assessments (including vital |
| Immucothel ®) | signs, ECGs, safety laboratory |
| To investigate the safety and tolerability of | parameters, and AEs) |
| remibrutinib administered as 100 mg b.i.d. for | All safety assessments (including vital |
| up to 35 days in healthy participants | signs, ECGs, safety laboratory |
| To explore the safety and tolerability of the | parameters, and AEs) |
| vaccinations administered to healthy | PK parameters: AUCtau (Day 15 |
| participants receiving remibrutinib | only), AUClast, Cmax, Tmax |
| To assess the PK of remibrutinib at a 100 mg | |
| b.i.d. dose | |

### Study Design

### Overall Design

This randomized, double-blind, placebo-controlled study has a parallel group design. Approximately 90 healthy female of non-childbearing potential and male participants are randomized to any of the three treatment groups in order to achieve a minimum of 72 evaluable completers considering an estimated drop-out rate up to 20%. The study will consist of a 28-day screening period, a 43-day treatment period, followed by a Study Completion evaluation (Day 57) within two weeks after last study drug administration. A safety follow-up call is performed approximately 30 days after the last study drug administration (Day 73). Participants are domiciled on Days-1 to 1 and Days 14-17. In total, the maximum study duration for each participant is about 85 days.

The impact of concomitant and interrupted remibrutinib treatment scenarios for Influenza/Pneumovax® 23 and Immucothel® is evaluated with reference to placebo.

### Study Conduct

### Screening & Baseline

Participants who meet the eligibility criteria at screening will be admitted to baseline evaluations on Day-1. All baseline safety evaluation results must be available prior to first dosing. At baseline, participants are randomized to one of the three treatment groups described below.

### Treatment

All participants receive study drug (remibrutinib 100 mg or placebo b.i.d.) from Day 1 until Day 42 and return to the clinic for End of Treatment visit at day 43. All participants also receive the quadrivalent Influenza vaccine, the PPV-23 vaccine and the KLH neoantigen vaccine on Day 15. Vaccinations should occur 3 hours after study drug administration.

During clinical visits and during domiciliation (Days-1 to 1 and Days 14-17), participants are administered the study drug by the study personnel at the clinic. Upon discharge from clinical visits during the Treatment period, study drug is provided to the participants for self-administration at home, along with the medication diary.

Safety assessments will include physical examinations, ECGs, vital signs, standard clinical laboratory evaluations (hematology, blood chemistry, urinalysis) adverse event and serious adverse event monitoring.

Multiple blood samples to assess remibrutinib pharmacokinetics will be drawn from all participants on Day 8, Day 15 and on Day 36.

### Group A (Concomitant Remibrutinib Treatment):

Participants will receive placebo (b.i.d.) from Days 1-7, followed by treatment with remibrutinib (100 mg b.i.d.) on study Days 8-15 to achieve PK/PD steady state, prior to administration of the three vaccines on Day 15. Participants will continue to receive remibrutinib (100 mg b.i.d.) until Day 42.

### Group B (Interrupted Remibrutinib Treatment):

Participants will be treated with remibrutinib 100 mg b.i.d from Day 1-7 to achieve PK/PD steady state conditions, followed by placebo (b.i.d.) administration from Day 8-28 and will be administered the three vaccines on Day 15. Treatment with remibrutinib 100 mg b.i.d. will be re-initiated treatment from Day 29 to 42.

### Group C (Placebo):

Participants in Group C will receive placebo (b.i.d) from Day 1-42 and will be vaccinated with the 3 vaccines on Day 15 under placebo conditions.

### Key Inclusion Criteria

Signed informed consent must be obtained prior to participation in the study.

Healthy, or mildly obese but otherwise healthy, male and non-childbearing potential female participants aged 18 to 55 years (inclusive).

Participants should be in good health as determined by past medical history, physical examination, vital signs, ECG, and laboratory tests at Screening and Baseline visit as indicated.

At Screening and Baseline, vital signs (systolic and diastolic blood pressure and pulse rate) will be assessed in the sitting position and again (when required by the assessment schedule) in the standing position. Sitting vital signs (after sitting 3 minutes) should be within the following ranges:
Tympanic body temperature of 35.0 to 37.5° C.
Systolic blood pressure (SBP) of 90 and 139 mmHg (inclusive).
Diastolic blood pressure of (DBP) 50 and 89 mmHg (inclusive).
Pulse rate of 45 and 90 bpm (inclusive).

Participants must weigh at least 50 kg to participate in the study and must have a body mass index (BMI) within the range of 18 to 34.9 kg/m2.

Participants must be willing to remain at the clinical site as required by the protocol and to comply with the requirements/instructions outlined in the ICF.

Able to read, speak, and understand the local language, to understand and comply with the requirements of the study.

Key Exclusion criteria

1. Use of other investigational drugs within 5 half-lives or 30 days prior to first dosing, whichever is longer.

2. Current evidence or past medical history of clinically significant ECG abnormalities or a family history (grandparents, parents, and siblings) of a prolonged QT interval syndrome or other abnormalities in cardiac conduction, history of additional risk factors for Torsade de Pointes (TdP) (e.g. heart failure, hypokalemia) and/or known history or current clinically significant arrhythmias. Abnormal ECG defined as PR >220 msec, QRS complex>120 msec, for males and females QTcF >450 msec, or any other morphological changes, other than early repolarization, nonspecific S-T or T-wave changes.

3. History or presence of malignancy of any organ system (other than localized basal cell carcinoma of the skin or in-situ cervical cancer), treated or untreated, within the past 5 years, regardless of whether there is evidence of local recurrence or metastases.

4. History or presence of any clinically significant disease of any major system organ class including (but not limited to) cardiovascular, pulmonary, metabolic, hepatic, renal, hematologic, endocrine, neurological or psychiatric diseases which had not resolved within two weeks prior to initial dosing

5. Hypersensitivity to remibrutinib or drugs from the same compound class or its excipients.

6. Any contraindication for the use of the Pneumovax 23, influenza or KLH vaccine including any acute infection, fever or hypersensitivity reactions or known hypersensitivity to any relevant component of the vaccines to be administered in this study (e.g., hen's egg or shellfish/KLH).

7. History of vaccination with the 2022-2023 seasonal influenza vaccine or known clinical diagnosis of influenza infection during the 2022-2023 influenza season prior to enrollment.

8. History of previous exposure or immunization with KLH.

1. A method of treating or preventing an IgE driven allergic reaction to one or more allergens, in a subject in need of such treatment or prevention, said method comprising administering a therapeutically effective dose of a BTK inhibitor, e.g. an irreversible BTK inhibitor, e.g. LOU064 or a pharmaceutically acceptable salt thereof.

2. The method according to claim 1, wherein the BTK inhibitor is LOU064 or a pharmaceutically acceptable salt thereof and wherein one or more allergens comprises or is food allergen.

3. The method according to claim 2 wherein food allergen is selected from peanut, tree nut, milk, wheat, egg, soy, sesame, fish and shelfish, particularly peanut.

4. The method of claim 2 wherein the therapeutically effective dose of LOU064 is from about 20 mg to about 200 mg daily.

5. The method of claim 4 wherein the therapeutically effective dose of LOU064 is from about 10 mg twice daily to about 100 mg twice daily.

6. The method of claim 5 wherein the therapeutically effective dose of LOU064 is about 10 mg twice daily.

7. The method of claim 5 wherein the therapeutically effective dose of LOU064 is about 25 mg twice daily.

8. The method of claim 5 wherein the therapeutically effective dose of LOU064 is about 100 mg twice daily.

9. (canceled)

10. (canceled)

11. (canceled)

12. The method of claim 2 wherein LOU064 is administered as a monotherapy.

13. The method of claim 2 wherein LOU064 is not administered concomitantly with a strong inhibitor of CYP3A, e.g. a strong inhibitor of CYP3A4.

14. The method of claim 2 wherein LOU064 is co-administered with a therapeutic agent.

15. The method of claim 14 wherein LOU064 is co-administered with corticosteroid and/or an immunosuppressor (e.g. inhaled corticosteroid), a leukotriene receptor antagonist (LTRA), a short-acting beta agonist (SABA) or a long-acting beta agonist (LABA).

16. The method of claim 2 wherein LOU064 is co-administered with an oral immunotherapy (OIT), sublingual immunotherapy (SLIT), epicutaneous immunotherapy (EPIT), preferably an OIT.

17. The method of claim 16 wherein LOU064 is an adjunct to oral immunotherapy (OIT),), sublingual immunotherapy (SLIT), epicutaneous immunotherapy (EPIT), preferably an OIT.

18. The method of claim 16 wherein the oral immunotherapy is a peanut protein (e.g. Palforzia™).

19. The method of claim 16 wherein LOU064 is administered starting at least 2 days (e.g. at least 2-14 days) prior to administration of the oral immunotherapy.

20. The method of claim 19 wherein LOU064, is administered during the escalation phase of the immunotherapy treatment.

21. The method of claim 2 wherein the method is preventing an IgE driven allergic reaction.

22. The method of claim 2 wherein the method is preventing anaphylaxis after accidental exposure to any allergens (e.g. food allergen).

23. The method of claim 21 wherein LOU064 achieves maximal prevention after a minimum of 2 days (e.g. after 2-14 days, preferably after 2-7 days) of treatment.

24. The method of claim 2 wherein the patient is selected according to one or more of the following criteria:
(e) Male and female patients, 6 years of age or older (e.g. 6 to 11 years of age, 12-17 years of age or 18-55 years of age);
(f) Documented medical history of allergy to foods, including but not limited to peanuts, tree nuts, wheat, egg, milk, soy, fish and shellfish;
(g) Positive allergen-specific IgE (e.g. peanut sigE≥6 kUA/L at Screening); and
(h) Skin prick test positive for allergen to which patient is allergic (e.g. defined as the average diameter (longest diameter and mid-point orthogonal diameter) ≥4 mm wheal compared to the negative control).

25. (canceled)

26. (canceled)

27. The method of claim 2 wherein at least one of the following applies:
d. At least 90% of the treated patients do not exhibit an allergic reaction upon food challenge with 600 mg of peanut protein
e. At least 90% of the treated patients do not exhibit an allergic reaction upon food challenge with 1000 mg of peanut protein
f. At least 80% of the treated patients do not exhibit an allergic reaction upon food challenge with 3000 mg of peanut protein;
after up to 4 weeks of treatment with LOU064 (e.g. after 1 week, or after 2 weeks, or after 3 weeks or after 4 weeks).

28. The method of claim 2 wherein the patient achieves a reduction from baseline in the total domain score FAQLQ of 0.45-0.5.

29. The method of claim 2 wherein the patient achieves a reduction from baseline in the total domain score FAIM.

30. The method of claim 2 wherein in a double-blind placebo-controlled food challenge with 600 mg of an allergen (e.g peanut allergen), the observed difference in the responder rate between the treated and non-treated patients is superior to 35%, wherein the responder rate is defined as no more than a mild response to 600 mg oral food challenge.

31. The method of claim 2 wherein by week 12 or by week 24 of treatment the levels of alanine aminotransferase (ALT), aspartate aminotransferase (AST), and lipase do not change by more than 10% as compared to the baseline level at the start of therapy.

32. (canceled)

33. (canceled)

34. (canceled)

35. (canceled)

36. (canceled)

37. (canceled)

38. (canceled)

39. (canceled)

40. (canceled)

41. (canceled)
